(19) United States
(12) Patent Application Publication
(10) Pub. No.: US 2024/0315632 A1
(43) Pub. Date: Sep. 26, 2024

(54) PREDICTING ERRONEOUS CLASSIFICATIONS OF PHYSIOLOGICAL PARAMETER SEGMENTS

(71) Applicants: Johannes de Bie, Monte San Pietro (IT); Ricardo Salinas-Martinez, Bologna (IT); Nicoletta Marzocchi, Bologna (IT); Frida Sandberg, Lund (SE)

(72) Inventors: Johannes de Bie, Monte San Pietro (IT); Ricardo Salinas-Martinez, Bologna (IT); Nicoletta Marzocchi, Bologna (IT); Frida Sandberg, Lund (SE)

(21) Appl. No.: 18/614,411

(22) Filed: Mar. 22, 2024

Related U.S. Application Data
(60) Provisional application No. 63/454,184, filed on Mar. 23, 2023.

(51) Int. Cl. A61B 5/361; A61B 5/00; A61B 5/256; A61B 5/271; A61B 5/353; G16H 40/67; G16H 50/20

(52) U.S. Cl. CPC A61B 5/361; A61B 5/256; A61B 5/271; A61B 5/353; A61B 5/6802; G16H 40/67; G16H 50/20; A61B 2562/222

(57) ABSTRACT

An example method includes identifying data indicative of measurements of a physiological parameter of a subject over a time period. The data is divided into multiple segments including a first segment and a second segment. The method further includes determining a first label indicating whether the first segment is indicative of a condition; generating a first image that is indicative of the first segment; and determining, by inputting the first image into a trained machine learning (ML) model, that the first label is inaccurate. The method additionally includes determining a second label indicating whether the second segment is indicative of the condition; generating a second image that is indicative of the second segment; and determining, by inputting the second image into the trained ML model, that the second label is accurate. The method additionally includes outputting an indication of whether the subject has the condition based on the second label.

### CROSS-REFERENCE TO RELATED APPLICATION

This application claims priority to U.S. Provisional Patent Application No. 63/454,184 filed on Mar. 23, 2023, which is incorporated herein by reference in its entirety as if fully set forth herein.

### TECHNICAL FIELD

This application relates generally to identifying a condition indicated by samples of a physiological parameter, and more particularly, to determining whether the condition will be misidentified based on features of the samples.

### BACKGROUND

Atrial fibrillation (AF) is a cardiovascular health condition affecting 2-3% of the population in Europe and in the United States (US). This arrhythmia is not life threatening by itself but if not treated adequately, it may lead to severe consequences such as stroke, heart failure, and death. Therefore, guidelines for the diagnosis and management of AF typically recommend electrocardiogram (ECG) screening on high-risk patients with a CHA2DS2-VASc score higher than zero in men and higher than one in women. The CHA2DS2-VASc score depends on patient risk factors including congestive heart failure, hypertension, age greater than 75 years, age between 65 and 74 years, biological sex, diabetes, stroke, and vascular disease.

Screening for AF can be performed either intermittently or continuously with surface ECG monitoring. Nowadays, intermittent screening is usually performed by pulse palpitations, hand-held devices, or wearable devices while continuous long-term monitoring is done with Holter recorders, ECG patches, or with telemetry systems in health centers. A recent study on 994 patients has shown that continuous 7-days ECG monitoring allows identification of AF patients 1.4-times more than 60-seconds intermittent ECG monitoring during 3-weeks. Similarly, studies have concluded that that paroxysmal AF is detected 2.5-times more with continuous 2-weeks ECG monitoring than with 30-second intermittent ECG monitoring during 2-weeks. These findings suggest that continuous long-term ECG monitoring is an effective method for detecting paroxysmal AF-episodes.

Mechanisms of AF are complex and not yet completely understood, which makes AF difficult to detect. In fact, some commercial algorithms for AF detection in continuous ECG recordings have set a minimum detection threshold. Documentation for the Philips Healthcare ST and Arrhythmia algorithm (Philips-Arrh) reports performance only for AF episodes longer than one minute. Specifications for the GE Healthcare AFib detection algorithm state that the algorithm produces an AF alarm to identify the onset of the episode within two minutes of presentation to the algorithm. These algorithms perform detection based on rhythm and P wave analysis allowing accurate detection of paroxysmal AF.

Paroxysmal AF longer than 30 seconds is associated with arrhythmogenic remodeling and contributes to a generalized prothrombotic state when left untreated. However, less is known about the prevalence and possible repercussions of brief atrial fibrillation (BAF) as many algorithms and studies for AF detection are limited to episodes longer than 30 seconds. The terms “brief atrial fibrillation,” “BAF,” “short AF,” “occult AF”, and “micro AF,” as used herein, may refer to AF episodes shorter than 30 seconds that are diagnosed from surface ECG recordings. Two preliminary studies on prevalence of BAF manually analyzed continuous long-term ECG recordings from stroke patients, and showed that 72% and 85% of the first diagnosed AF episodes are shorter than 30 seconds. Additionally, a recent study has suggested that BAF episodes gradually progress into longer AF episodes, highlighting the importance of early detection and treatment.

In order to facilitate the detection of BAF episodes, some researchers have proposed methodologies for automatic detection of BAF in continuous ECG recordings based on rhythm irregularity, P-wave absence, rhythm irregularity and P-wave absence, and time-frequency features of the raw ECG.

### SUMMARY

Computer-based classifiers can be utilized to identify the presence or absence of a condition indicated by physiological parameter data. However, in some cases, classifiers misidentify the presence or absence of the condition in the data. For instance, the data may include one or more artifacts or other types of noise that prevent accurate identification of the condition. This problem can be particularly acute in examples in which the physiological parameter data is obtained from long-term recordings by a wearable sensor of a patient, where the patient may be moving as the sensor is acquiring the physiological parameter data. For example, the sensor may be incorporated into a wearable device that monitors the patient over the course of hours or days.

Various implementations of the present disclosure relate to techniques for determining whether a given classifier is likely to misidentify a condition (or the absence of the condition) in physiological parameter data. According to various cases, a predictive model can be utilized to identify one or more features of the physiological parameter data that are likely to result in misclassification by the classifier. For example, the predictive model may identify that the data includes a motion artifact that would prevent the classifier from accurately identifying the presence or absence of the condition in the data. In some cases, these techniques can be relied upon to ignore erroneous classifications by the classifier. For example, a clinician relying on the classifier may ignore (or may never be presented with) classifications from data that the predictive model identifies is likely to be misclassified, thereby enhancing the specificity of the classifier. In various implementations, the classification performed by the classifier can be relevant to a diagnosis of the subject by the clinician. Therefore, techniques described herein can enhance the diagnostic accuracy of evaluations that consider classifications performed by computer-based classifiers.

In some cases, techniques described herein can be utilized to enable a resting electrocardiogram (ECG) classifier to accurately identify a condition in ECG segments that are obtained from a non-resting patient. For example, an ECG of a patient can be obtained over the course of hours by a wearable device, such as a Holter monitor. In some cases, the ECG can be divided into individual segments with limited durations (e.g., 5, 10, or 30 seconds). A computer system executing a classifier may automatically identify whether each segment is indicative of a condition, such as AF. However, due to the presence of a motion artifact in a particular segment, the computer system may return an erroneous classification of AF in the particular segment. In particular instances, a predictive model can additionally receive an input based on the particular segment. The predictive model, for instance, may flag the particular segment as being likely to be misclassified. Based on the flag, the computer system may refrain from returning the erroneous classification of the particular segment to a relying cardiologist. Thus, even though the AF classifier is sensitive to motion artifact and other sources of noise, accurate classifications of the AF classifier may be relied upon for diagnostic purposes.

Various types of predictive models that discriminate between reliable and unreliable physiological parameter data are encompassed by this disclosure. In some cases, a predictive model includes a machine learning (ML) model, such as a convolutional neural network (CNN). The ML model, for instance, is trained using a supervised learning process. For example, parameters in the ML model can be optimized based on training data that includes various segments of physiological parameter data obtained from multiple subjects, classifications of the segments by the classifier (which may or may not be accurate), as well as true classifications of the segments. By optimizing the parameters, the ML model can be trained to identify features of data that will lead the classifier to misclassify the segments.

### DESCRIPTION OF THE FIGURES

The following figures, which form a part of this disclosure, are illustrative of described technology and are not meant to limit the scope of the claims in any manner. Some of the figures submitted herein may be better understood in color. Applicant considers the color versions of the figures as part of the original submission and reserves the right to present figures images of the drawings in later proceedings.

FIG. 1 illustrates an example environment for accurately classifying segments of an ECG.

FIG. 2 illustrates example signaling associated with a classifier.

FIG. 3 illustrates example signaling associated with a discriminator.

FIG. 4 illustrates example signaling for training a predictive model.

FIG. 5 illustrates example signaling associated with a CNN.

FIG. 6 illustrates an example of a convolutional block in a neural network.

FIG. 7 illustrates an example process for training a discriminator to identify whether a classifier will accurately classify input data.

FIG. 8 illustrates an example process for determining whether a classifier will accurately classify input data.

FIG. 9 illustrates an example process for classifying whether input data is indicative of a condition.

FIG. 10 illustrates at least one example device configured to enable and/or perform the some or all of the functionality discussed herein.

FIG. 11A illustrates an example schematic indicating how databases were used in the development and testing of a proposed AF detector of an experimental example.

FIG. 11B illustrates an example of a generated ECG recording showing the onset and offset of an example 10-second AF episode.

FIG. 12A illustrates an example schematic indicating how ECGs are classified as AF or non-AF by a computerized resting ECG algorithm.

FIG. 12B illustrates (top) a segment correctly labeled as AF by the Cl-algorithm and the CNN, and (bottom) a segment incorrectly labeled as AF by the Cl-algorithm identified as a false detection by the CNN.

FIG. 13 illustrates the architecture of the CNN used in the experimental example.

FIG. 14 illustrates an example of episode sensitivity for BAF detection of the of a resting ECG algorithm on the generated ECG recordings with different durations of AF.

### DETAILED DESCRIPTION

Various implementations of the present disclosure will be described in detail with reference to the drawings, wherein like reference numerals present like parts and assemblies throughout the several views. Additionally, any samples set forth in this specification are not intended to be limiting and merely set forth some of the many possible implementations.

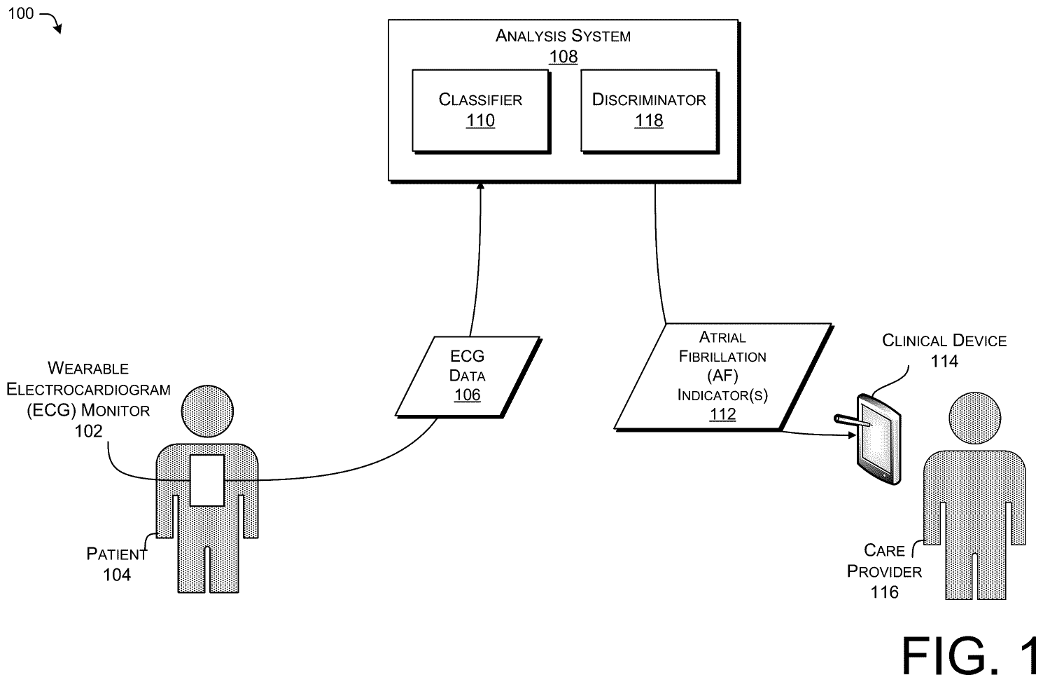

FIG. 1 illustrates an example environment 100 for accurately classifying segments of an ECG. The environment 100 includes a wearable ECG monitor 102 that is configured to monitor a patient 104. The patient 104 may be a human subject. Although not illustrated in FIG. 1, other types of human and non-human subjects can be monitored using various implementations of the present disclosure. Further, ECG is one of numerous examples of physiological parameters that can be analyzed using various techniques described herein.

The wearable ECG monitor 102 is an external ECG monitor. The wearable ECG monitor 102, for instance, includes electrodes that are in contact with the skin of the patient 104. In some cases, the electrodes are adhered to the skin of the patient 104 using an electrically conductive adhesive. According to various examples, at least one first electrode among the electrodes is adhered to the skin on a first side of the heart of the patient 104, and at least one second electrode among the electrodes is adhered to the skin on a second side of the heart of the patient 104. The wearable ECG monitor 102 may include at least one electrical sensor configured to detect electrical signals relative to combination of two or more electrodes. For instance, the electrical sensor(s) is configured to detect a relative voltage between the first electrode and the second electrode. The heart of the patient 104 may output a changing electrical potential as the heart beats. Therefore, the electrical signals detected by the electrical sensor(s) may be used to generate the ECG.

According to various implementations, the ECG includes multiple leads. As used herein, the term “lead,” and its equivalents, refers to an electrical signal relative to a combination of two or more electrodes detected over time or the waveform that is representative of the electrical signal. In various cases, the ECG includes twelve leads detected relative to twelve combinations of two or more electrodes adhered to the skin of the patient 104. In some cases, the ECG detected by the wearable ECG monitor 102 includes fewer than twelve leads, such as ten leads, six leads, three leads, or a single lead.

In various cases, the wearable ECG monitor 102 is configured to be worn by the patient 104. The wearable ECG monitor 102 may include one or more straps, buckles, elastic bands, or other physical fasteners configured to attach to the patient 104. In some cases, the wearable ECG monitor 102 includes a housing attached to a belt of the patient 104, wherein the housing includes the electrical sensor(s) and/or additional circuitry. The electrodes, for instance, may be electronically coupled to a circuit disposed in an interior space of the housing by wires or other electrically conductive structures. In some examples, the wearable ECG monitor 102 includes a vest configured to fasten to the patient 104.

The wearable ECG monitor 102 may be configured to detect an ECG of the patient 104 over the course of an extended time period. For example, the wearable ECG monitor 102 is configured to detect the ECG for a duration that is greater than 1 minute, greater than 10 minutes, greater than 30 minutes, greater than one hour, greater than five hours, greater than 10 hours, or greater than 24 hours. In some implementations, the duration is less than three weeks.

In various cases, the wearable ECG monitor 102 detects the ECG at a sampling rate that is greater than 100 Hz. For example, the ECG is sampled at 200 Hz, 300 Hz, 400 Hz, 500 Hz, or 1 kHz. Accordingly, individual leads of the ECG detected by the wearable ECG monitor 102 include numerous individual measurements over time. In some cases, each lead can be represented as a waveform.

The wearable ECG monitor 102 may generate ECG data 106 and output the ECG data 106 to an analysis system 108. The ECG data 106 indicates the ECG detected by the wearable ECG monitor 102 over the duration. In some implementations, the ECG data 106 includes multiple channels corresponding to the multiple leads of the ECG detected by the wearable ECG monitor 102. In some cases, the wearable ECG monitor 102 transmits the ECG data 106 in multiple data packets. For instance, the ECG monitor 102 may stream the ECG data 106 to the analysis system 108 in real-time or substantially in real-time. In some cases, the ECG monitor 102 outputs the ECG data 106 after the duration, such as after the ECG data 106 is generated.

The analysis system 108 may be configured to analyze the ECG data 106. The analysis system 108, in various implementations, may be implemented on at least one computing device. For instance, at least one processor may be configured to perform the functions of the analysis system 108. In some cases, instructions for performing the functions are stored in memory of the computing device(s).

According to various cases, the analysis system 108 includes a classifier 110. The classifier 110 is configured to detect whether the ECG data 106 is indicative of a condition. In various implementations, the classifier 110 is configured to detect whether the ECG data 106 indicates that the patient 104 has atrial fibrillation (AF) or some other heart rhythm-related pathology. According to particular cases, the classifier 110 is configured to detect whether the ECG data 106 indicates brief episodes of AF (BAF). As used herein, the terms “brief episodes of AF,” “BAF,” and their equivalents, refer to an episode of AF that last for less than 30 approximately seconds. In some implementations, the classifier 110 is configured to determine an amount of the ECG data 106 that is indicative of the condition. For instance, the classifier 110 may determine a percentage of the duration of the ECG data 106 that is indicative of AF.

In some implementations, the classifier 110 is configured to divide the ECG data 106 into multiple segments and to determine whether each of the segments is indicative of the condition. For example, an individual segment may correspond to a time interval that is significantly shorter than the duration of the ECG data 106. In some cases, the time interval is one or more seconds long, such as a length in a range of 1 to 30 seconds. The classifier 110, for instance, may individually identify whether the condition is present in each of the multiple segments.

The classifier 110 may apply various techniques to identify the condition. In some cases, the classifier 110 converts the ECG data 106 (or segments of the ECG data 106) into the frequency domain by performing a Fourier transform. The condition, for instance, may be associated with a predetermined spectral signature. In various cases, the classifier 110 identifies whether the predetermined spectral signature is present in the frequency-domain spectra of the ECG data 106 (or segments).

Other types of analysis can also be performed. U.S. Pub. No. 2022/0273224, which is hereby incorporated by reference, describes various techniques for detecting BAF in data representing an ECG. In some cases, the classifier 110 identifies beat-to-beat intervals in segments of the ECG data 106, identifies whether p waves are present in the segments of the ECG data 106, and classifies the segments of the ECG data 106 as including or not including BAF based on the beat-to-beat intervals and whether the p waves are present. For instance, the classifier 110 may generate processed data by detecting and assigning counters and/or time stamps to each heartbeat (e.g., QRS complex) in the ECG data 106. The classifier 110 may divide the processed data into overlapping segments. The classifier 110 may truncate the processed data by removing amplitudes above a first threshold and below a second threshold. The classifier 110 may identify one or more segments including heartbeats in the truncated, processed data that indicate the presence or absence of P waves within the data. The classifier may generate an electrocardiomatrix (ECM) based on the segment(s). In some examples, the classifier inputs the ECM into a predictive model (e.g., a CNN) trained to identify the presence of AF. In some cases, the classifier 110 may apply the VERITAS-REST algorithm (or a different resting-ECG algorithm) to the ECG data 106 or segments.

In some cases, however, the classifier 110 may erroneously classify the segments of the ECG data 106. For example, the classifier 110 may misclassify a segment that indicates the condition as a segment that does not indicate the condition (also referred to as a “false negative”). In some cases, the classifier 110 may misclassify a segment that does not indicate the condition as a segment that does indication the condition (also referred to as a “false positive”). Several features of the ECG data 106 may cause misclassifications by the classifier 110. For example, the patient 104 may have moved around (e.g., walked, run, danced, or participated in other types of movements) while the wearable ECG monitor 102 was detecting the ECG of the patient 104. These movements may have caused artifact in the ECG data 106 that can be referred to herein as “motion artifact.” Other types of features of the ECG data 106 may result in misclassifications. For example, a relatively rapid heart rate (which could be the result of strenuous exercise) may be misclassified as AF. In some cases, noise from mains power in an indoor environment may create noise in the ECG data 106. The types of features that are likely to confuse the classifier 110 may be dependent on the techniques applied by the classifier 110 to identify the condition in a given segment of the ECG data 106.

The analysis system 108 may output AF indicators 112 to a clinical device 114 based on the classification(s) performed by the classifier 110. The AF indicator(s) 112, for example, indicate whether the ECG data 106 is indicative of the condition, whether segments of the ECG data 106 are indicative of the condition, or a portion (e.g., at least one segment) of the ECG data 106 that is indicative of the condition. The clinical device 114 may be a computing device operated by a care provider 116. For example, the clinical device 114 includes a mobile device, smart phone, tablet computer, laptop, desktop computer, IoT device, or some other type of computing device. The care provider 116 may be responsible for diagnosing and/or treating the patient 104. For example, the care provider 116 may be a physician (e.g., a cardiologist, neurologist, or the like), a physician's assistant, a resident, a medical student, a nurse, or medical technician. In various cases, the clinical device 114 may output the AF indicator(s) 112 to the care provider 116. The AF indicator(s) 112 may indicate whether the classifier 110 determines that the ECG data 106 is indicative of the condition, whether the classifier 110 determines that the segments are indicative of the condition, how many segments are determined by the classifier 110 to be indicative of the condition, the segments that are determined by the classifier 110 to be indicative of the condition, or any combination thereof.

However, if the AF indicator(s) 112 is based on an erroneous classification by the classifier 110, then the care provider 116 may erroneously diagnose or treat the patient 104. If AF is associated with a heightened risk of another pathology (e.g., stroke), it may be a standard of practice to diagnose a prophylactic treatment to reduce the risk of the other pathology to patients with AF. If the patient 104 has AF, but the AF is not correctly reported by the AF indicator(s) 112, then the care provider 116 may refrain from administering the clinically indicated prophylactic treatment, thereby not treating the heightened risk of stroke of the patient 104. In contrast, if the patient 104 does not have AF, but the AF indicator(s) 112 erroneously indicate that the patient 104 has AF, then the care provider 116 may administer the prophylactic treatment unnecessarily.

Thus, the accuracy of the AF indicator(s) 112 can have a significant impact on the health of the patient 104. Further, because the ECG data 106 is generated by the wearable ECG monitor 102 and more likely to include motion artifact and other features that can cause the classifier 110 to misclassify the ECG segments, misclassification of the ECG data 106 by the classifier 110 is more likely than misclassification of data generated by non-wearable sensor devices.

Various implementations of the present disclosure address these and other problems by utilizing a discriminator 118. The discriminator 118, for instance, is part of the analysis system 108. According to various cases, the discriminator 118 is configured to identify at least one segment of the ECG data 106 that is misclassified, or otherwise will be misclassified by the classifier 110. For instance, the discriminator 118 indicates whether an individual segment of the ECG data 106 will result in a false positive AF indicator 112 or a false negative AF indicator 112. In various implementations, an individual ECG segment is input into the discriminator 118 and the discriminator 118 outputs an indication of whether the classifier 110 will accurately classify the segment (e.g., “true” or “false”). In some cases, the AF indicator 112 generated by the classifier 110 based on the segment is also input into the discriminator 118.

In particular examples, the classifier 110 classifies segments of the ECG data 106 as depicting AF. Due to various sources of uncertainty, the segments may include at least one false positive segment and at least one true positive segment. However, if the care provider 116 is presented with the false positive segment(s), the care provider 116 may inaccurately diagnose the patient 104. The discriminator 118, however, may identify the false positive segment(s), and prevent the analysis system 108 from outputting the false positive segment(s) in the AF indicator(s) 112. In various cases, the classifier 110 may have relatively high sensitivity, such that there is a low likelihood that the classifier 110 will produce false negatives by incorrectly classifying segments of the ECG data 106 as omitting AF when they, in fact, depict AF. Thus, in various cases, the discriminator 118 may exclusively analyze segments of the ECG data 106 that are classified, by the classifier 110, as depicting AF.

In some examples, however, the classifier 110 may be less sensitive and may incorrectly classify a significant number of segments of the ECG data 106 as omitting AF. In some cases, the segments classified as non-AF are analyzed by the discriminator 118, which may be configured to identify at least one of the segments that is incorrectly classified as non-AF (false negatives). In various cases, the discriminator 118 may cause the analysis system 108 to output the false negative segment(s) to the care provider 116. According to some implementations, the discriminator 118 includes two models, one model for distinguishing false positive and true positive segments of the ECG data 106, and another model for distinguishing false negative and true negative segments of the ECG data 106.

In some implementations, the discriminator 118 includes at least one statistical model configured to determine whether segments are likely to be accurately classified by the classifier 110. According to various cases, the discriminator 118 includes at least one machine learning (ML) model. As used herein, the terms “machine learning model,” “ML model,” and their equivalents, may refer to a computer model that is designed to be optimized (or “trained”) to identify patterns or other types of features in a data set. For instance, the discriminator 118 includes one or more first ML models configured to distinguish false positive and true positive segments of the ECG data 106 and/or one or more second ML models configured to distinguish false negative and true negative segments of the ECG data 106.

In various implementations, the discriminator 118 includes at least one convolutional neural network (CNN). A CNN is a type of artificial neural network configured to adaptively learn spatial hierarchies of features from input datasets. In various cases, the input datasets are images. A CNN includes various convolutional layers that are defined according to various parameters. These parameters can be shared across locations in an input image, allowing the network to learn location-invariant features, such as edges or textures. An individual convolutional layer applies an image filter (also referred to as a “kernel”) to an input image. For instance, the image filter is cross-correlated or convolved with the input image. The filter is defined according to various parameters, such as numbers. Each convolutional layer may be defined according to a kernel size, an output channel, and a stride size. In various implementations, the kernel of a convolutional layer is defined in two dimensions. The kernel (or “filter”) of a given convolution block or layer is convolved and/or cross-correlated with an input to the convolutional layer. A 2D kernel can be represented as a 2D matrix, in some implementations. For instance, the kernel size may be 2×2, 3×3, 4×4, 5×5, 6×6, or the like. In some cases, a single CNN may utilize kernels of different sizes.

The stride size of a convolutional layer corresponds to the distance between pixels that are convolved and/or cross-correlated with the kernel at a given time. A stride size of 1 indicates that the kernel is convolved and/or cross-correlated with adjacent pixels in the input. A stride size of 2 indicates that the kernel is convolved and/or cross-correlated with pixels in the input that are spaced apart by one pixel. In various implementations, convolutional layers have strides of 1, 2, 3, or the like. In some cases, a CNN may utilize strides of different sizes.

In some cases, the CNN is trained by optimizing the parameters of the filters in the convolutional layers. For instance, during training, the values of the kernels (e.g., the numbers within the matrices that define the kernels) may be optimized based on training data. These parameters can be optimized from training data through backpropagation and/or gradient descent.

In some implementations, the convolutional layers of a CNN are included in convolutional blocks. As used herein, the term “block,” and its equivalents, may refer to one or more layers connected in series. A convolutional block, for instance, may include a convolutional layer, a normalization layer, and an activation layer.

A CNN may also include various pooling layers, which are used to reduce the spatial size of the feature maps, leading to a reduction in the number of parameters in the network and making it more computationally efficient. Fully connected layers can be used to make the final classification decision of the CNN based on the learned features from the previous layers. A max pooling layer is an example of a pooling layer.

In some cases, a CNN includes multiple convolutional blocks. In various implementations, a first convolutional block in the CNN transforms an input image into an intermediary image, referred to as a “feature map” or “activation map,” which is subsequently input into a second convolutional block in the CNN. Because the output of the first convolutional block is an input of the second convolutional block, the first and second convolutional blocks may be referred to as being “in series.”

According to some examples, the input of a CNN may have different dimensions than an output of the CNN. For example, an image may be input into a CNN and a Boolean value (e.g., “true” or “false”) may be output by the CNN. In some examples, the dimensions of the input image can be reduced using a dilation rate of greater than one, a step size of greater than one, or any combination thereof.

In various cases, the discriminator 118 converts input data (e.g., at least one segment of the ECG data 106) into a representation (e.g., an image). In some examples, the discriminator 118 generates a spectrogram of the input data. A spectrogram is an image-based representation of frequency components of data with respect to time. For instance, the discriminator 118 may generate the spectrogram by performing a Fourier transform (e.g., a Fast Fourier Transform (FFT), if the input data is discrete) on the input data. In some cases, the Fourier transform is a short-time Fourier transform (STFT). In some examples in which the input data includes multiple channels, the discriminator 118 converts the input data into a single channel with each of the multiple channels corresponding to consecutive time intervals. The discriminator 118 may convert the single channel of the input data into the spectrogram. In various cases, one dimension (e.g., an x-axis) of the image represents time, another dimension (e.g., a y-axis) of the image represents frequency, and a third dimension (e.g., a color or z-dimension) of the image represents an amplitude of the input data at the corresponding time and frequency. In some examples, the discriminator 118 generates a representation from the input data using a Wavelet Transform Modulus Maxima (WTMM), a Constant-Q Transform (CQT), a Wigner-Ville Distribution (WVD), an Empirical Mode Decomposition (EMD), a scalogram, a wavelet transform, or any combination thereof. The representation generated by the discriminator 118 may be a two-dimensional (2D) image or a three-dimensional (3D) image, for instance.

The discriminator 118, in various examples, inputs the image into the ML model. Various convolutional layers, for instance, within the ML model may transform the image into a Boolean output, represented by “true” or “false.” In some cases, an output of “true” indicates that the classifier 110 will correctly identify the segment represented by the image, whereas an output of “false” indicates that the classifier 110 will incorrectly identify the segment represented by the image.

In various implementations, the analysis system 108 may selectively rely on the classifier 110 for segments that will be correctly classified, as determined by the discriminator 118. For example, if a first segment of the ECG data 106 is determined, by the discriminator 118, as being misclassified by the classifier 110, then the analysis system 108 will refrain from outputting a classification generated by the classifier 110 based on the first segment and/or will refrain from outputting the misclassified segment. However, if a second segment of the ECG data 106 is determined, by the discriminator 118, as not being misclassified by the classifier 110, then the analysis system 108 will output a classification generated by the classifier 110 based on the second segment among the AF indicator(s) 112. As a result, the clinical device 114 can receive accurate classifications of segments of the ECG data 106, without receiving inaccurate classifications of segments of the ECG data 106. The care provider 116 may therefore rely on accurate classifications of the ECG data 106, which can enhance the diagnostic accuracy and potential treatment options implemented by the care provider 116, thereby enhancing the health of the patient 104.

In a particular example, the patient 104 may be monitored by the wearable ECG monitor 102 over the course of a 24-hour period. The ECG data 106 includes discrete data representing the ECG of the patient 104 sampled over the course of the 24-hour period by the wearable ECG monitor 102. During a first time interval in the 24-hour period, the patient 104 is jogging, which produces a significant artifact in a first segment of the ECG data 106 corresponding to the first time interval. During a second time interval in the 24-hour period, the patient 104 is sitting down and watching a calm nature documentary, which produces minimal artifact in a second segment of the ECG data 106 corresponding to the second time interval.

The analysis system 108 receives the ECG data 106 and divides the ECG data 106 into multiple segments, including the first segment and the second segment. The discriminator 118 converts the first segment into a first image and uses a trained ML model to determine that the first segment will be misclassified by the classifier 110. This misclassification, for example, may be due to the artifact generated while the patient 104 was jogging. The discriminator 118 also converts the second segment into a second image and uses the trained ML model to determine that the second segment will be correctly classified by the classifier 110. In some cases, the classifications output by the classifier 110 are also used, by the discriminator 118, to determine whether the second segment has been correctly classified by the classifier 110.

In some cases, the classifier 110 classifies the first segment as depicting AF. However, this classification may be inaccurate due to the motion artifact in the first segment. In various examples, the discriminator 118 may analyze the first segment and determine that the first segment includes at least one feature (e.g., the motion artifact) that is likely to cause the classifier 110 to misclassify the first segment. According to examples of the present disclosure, the analysis system 108 may refrain from including the first segment in the AF indicator(s) 112, despite the classification performed by the classifier 110, due to the determination by the discriminator 118.

In various implementations, the classifier 110 determines that AF is present in the second segment. Accordingly, the AF indicator(s) 112 may indicate the classification generated by the classifier 110 with respect to the second segment. That is, the AF indicator(s) 112 may indicate that AF is present in the second segment. The clinical device 114, for instance, may visually output the second segment to the care provider 116 with a label indicating that AF is present. The care provider 116, in some implementations, may rely on the AF indicator(s) 112 to determine that the patient has AF. For example, the care provider 116 may tell the patient 104 that they have at least one risk factor for stroke.

According to various instances, the discriminator 118 may include a first ML model trained to distinguish between false positive and true positive segments, as well as a second ML model trained to distinguish between false negative and true negative segments. For example, the first ML model is trained with first training data that includes segments misclassified by the classifier 110 as depicting AF, when the segments do not actually depict AF. Further, the second ML model is trained with second training data that includes segments misclassified by the classifier 110 as not depicting AF, when the segments actually do depict AF. In various implementations, the first and second segments are analyzed by the first ML model of the discriminator 118. In contrast, a third segment of the ECG data 106 that is classified by the classifier 110 as not depicting AF may be input into the second ML model. The second ML model, in some cases, may indicate that the third segment is likely to be misclassified by the classifier 110. Accordingly, the analysis system 108 may output the third segment in the AF indicator(s) 112 for further analysis by the care provider 116.

In some implementations, a determination by the discriminator 118 that a segment is likely to be misclassified by the classifier 110 may trigger another level of analysis. For example, the classifier 110 may utilize a first technique for determining whether AF is present in various segments of the ECG data 106. Upon the discriminator 118 determining that the first segment is likely to be misclassified by the classifier 110, the analysis system 108 may input the first segment into a secondary classifier (not illustrated) that utilizes a second technique for determining whether AF is present in segments of the ECG data 106. For instance, the first technique may utilize a static, statistical model, whereas the second technique may utilize a ML model trained to determine whether AF is present in the segment. It may be computationally expensive for the analysis system 108 to analyze every segment of the ECG data 106 using the second technique. However, a balance between processing efficiency and accuracy can be obtained using the discriminator 118, which may be used to select a subset of segments for additional analysis by the secondary classifier.

Although FIG. 1 is described with respect to determining whether AF is indicated in an ECG, implementations of the present disclosure are not so limited. For example, a similar analysis system can be used to analyze other types of physiological parameters, such as a concentration of a component in a fluid sample obtained from the patient (e.g., a blood glucose level), a vital sign (e.g., a body temperature, a respiration rate, or a pulse rate), a blood oxygen saturation (e.g., a plethysmograph), a partial pressure of a gas in the airway of the patient (e.g., a capnograph), a blood pressure, an electroencephalogram (EEG), an electromyograph (EMG), or any combination thereof. Various other types of conditions besides AF can also be detected. For instance, implementations of the present disclosure can be utilized to detect ketoacidosis, sepsis, fever, apnea, seizure, or other types of arrhythmias besides AF (e.g., tachycardia, bradycardia, atrial flutter, ventricular tachycardia, supraventricular tachycardia, or an atrioventricular block).

In various cases, data can be generated by a wearable sensor measuring a physiological parameter of the patient 104. That data can be segmented and the discriminator 118 can be used to determine which, if any of the segments, will be misclassified by the classifier 110. The classifier, in turn, can classify at least some of the segments based on the presence or absence of physiological condition indicated by the segment(s). The analysis system 108 may output indications of the classifications of the physiological condition made by the classifier 110.

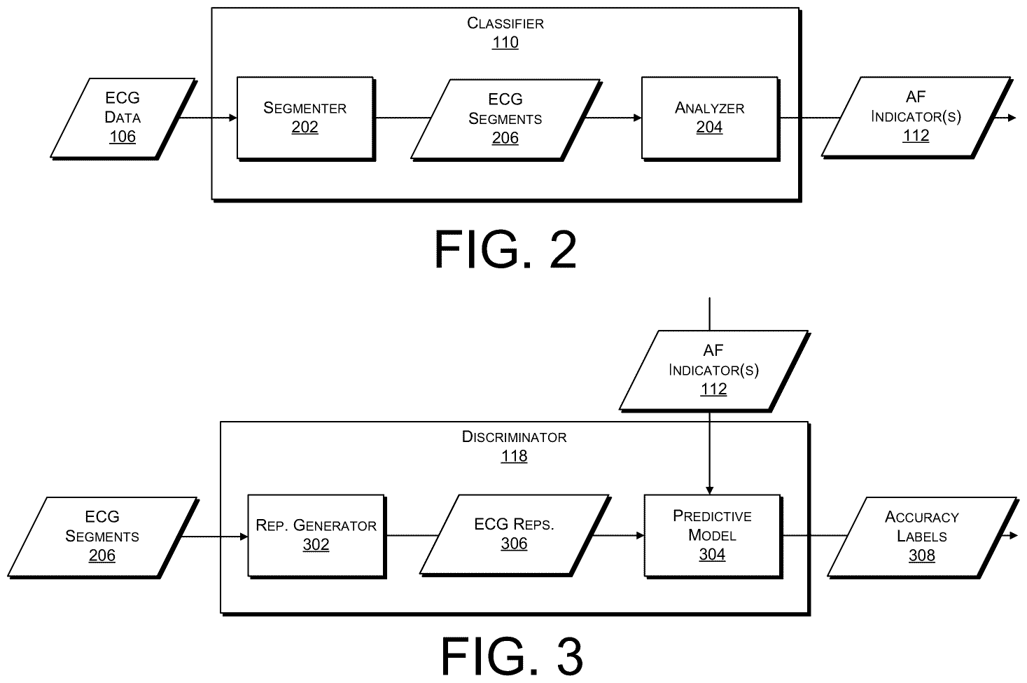

FIG. 2 illustrates example signaling associated with the classifier 110 described above with reference to FIG. 1. The classifier 110 may include a segmenter 202 and an analyzer 204. In general, the classifier 110 is configured to determine whether the ECG data 106 includes one or more features associated with a predetermined condition.

In various implementations, the segmenter 202 receives the ECG data 106. The ECG data 106, for instance, may include data representing an ECG of a patient that has been obtained over a relatively long measurement window, such as a measurement window that is greater than 1 minute. For instance, the measurement window can be 1 minute, 30 minutes, 1 hour, 6 hours, 12 hours, 24 hours, or 48 hours. The segmenter 202 may divide the ECG data 106 into ECG segments 206 representing time intervals that are shorter than the total measurement window of the ECG data 106. In various examples, the ECG segments 206 include portions of the ECG data 106 measured over time intervals that are less than 1 minute. For example, the ECG segments 206 can represent portions of the ECG data 106 obtained over time intervals with a length of 1 second, 5 seconds, 10 seconds, or 30 seconds. The ECG segments 206 may be overlapping or non-overlapping. For example, a first segment among the ECG segments 206 may represent measurements taken during 2:00:00 to 2:00:30, and a second segment among the ECG segments 206 may represent measurements taken during 2:00:15 to 2:00:45 (if overlapping) or 2:00:30 to 2:01:00 (if non-overlapping).

The analyzer 204 may be configured to identify whether a condition is present in the ECG segments 206. For instance, the analyzer 204 may determine whether AF (or BAF) is present in each of the ECG segments 206. Various algorithms can be performed by the analyzer 204 in order to classify the ECG segments 206. For example, the analyzer 204 may apply an algorithm suitable for a resting ECG analysis, such as VERITAS-REST. In various cases, the analyzer 204 outputs the AF indicator(s) 112 based on whether the condition is present in the ECG segments 206. In some implementations, the AF indicator(s) 112 represent classifications performed by the analyzer 204 on only a portion of the ECG data 106 segments generated by the segmenter 202.

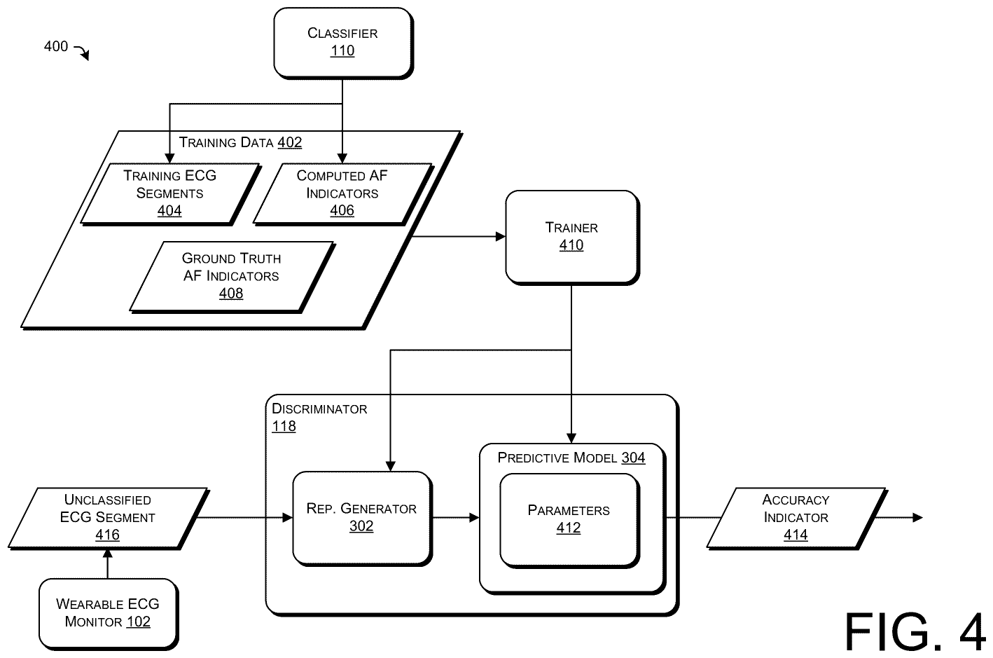

FIG. 3 illustrates example signaling associated with the discriminator 118 described above with reference to FIG. 1. The discriminator 118 includes a representation generator 302 and a predictive model 304. In various implementations, the discriminator 118 is configured to determine which, if any, of the ECG segments 206 will be misclassified by the analyzer 204 of the classifier 110 described above with reference to FIG. 2.

The representation generator 302 is configured to generate ECG representations 306 based on the ECG segments 206. For instance, the representation generator 302 may convert each of the ECG segments 206 into an individual representation among the ECG representations 306. The ECG representations 306, for instance, may represent frequency components of the ECG segments 206. In some cases, the ECG representations 306 include scalograms, spectrograms, WTMM representations, CQT representations, WVD representations, EMD representations, representations representing wavelet transforms of the ECG segments 206, or any combination thereof. In various examples, the representation generator 302 produces images that are representative of the ECG segments 206. That is, the ECG representations 306 may be images defined according to pixels.

The predictive model 304 is configured to determine which (if any) of the ECG segments 206 will be misclassified by the analyzer 204 of the classifier 110 based on the ECG representations 306 and the AF indicators 112. In various cases, the predictive model 304 includes an ML model, such as at least one CNN. Each ECG representation among the ECG representations 306 (and corresponding AF indicator 112 among the AF indicator(s) 112) can be input into the predictive model 304 individually. The predictive model 304 may generate a label among accuracy labels 308 based on each ECG representation among the ECG representations 306. Thus, the predictive model 304 generates the same number of accuracy labels 308 as there are ECG representations 306. In various cases, the accuracy labels 308 include Boolean values indicating that the respective ECG representations 306 will be correctly or incorrectly classified by the analyzer 204. In some implementations, the accuracy labels 308 further indicate one or more features of the ECG representations 306 leading to an incorrect classification by the analyzer 204. For instance, if one of the ECG representations 306 includes a motion artifact that will lead to a misclassification by the analyzer 204, the accuracy labels 308 may indicate the motion artifact in the ECG representation 306.

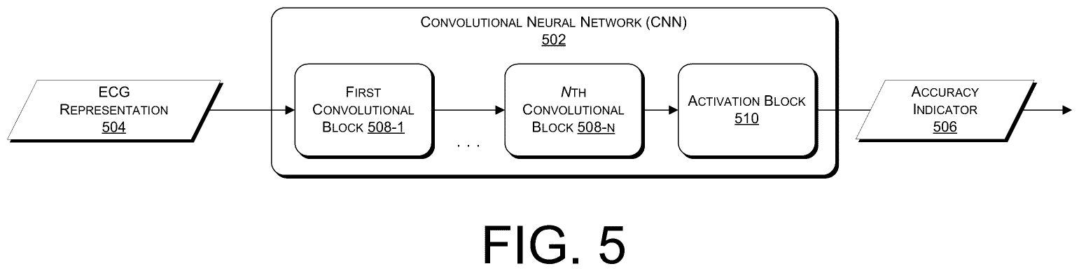

FIG. 4 illustrates example signaling 400 for training the predictive model described with reference to FIG. 3. The signaling 400, for example, is between the wearable ECG monitor 102, the classifier 110, the discriminator 118, and the predictive model 304 described above with reference to FIGS. 1 and 3.

The predictive model 304, in various implementations, is trained using training data 402. The training data 402, for example, includes multiple training ECG segments 404, computed AF indicators 406, and ground truth AF indicators 408. The training ECG segments 404 include segments of ECG data obtained from one or more subjects in a population. In some cases, the training ECG segments 404 are generated based on ECG data generated by the wearable ECG monitor 102, but implementations are not so limited. The training ECG segments 404 include segments with different quality levels. Some of the ECG segments 404, for example, include motion artifact or other types of noise. Some of the ECG segments 404 may indicate episodes of AF (e.g., BAF). Some of the ECG segments 404 may lack indications of any arrhythmias or pathologies. In various cases, the training ECG segments 404 indicate a variety of conditions, are obtained from a variety of subjects, and have a variety of signal qualities. At least some of the training ECG segments 404 may be generated by the classifier 110 (e.g., the segmenter 202 described above with reference to FIG. 2). In some implementations, the training ECG segments 404 can be substituted for ECG representations of the training ECG segments 404, such as representations generated by inputting the training ECG segments 404 into the representation generator 302 described above with reference to FIG. 3. In various implementations, at least some of the training ECG segments 404 are obtained from non-wearable sources, such as from ECG segments obtained from resting ECG recordings.

The computed AF indicators 406 include classifications of the training ECG segments 404 by the classifier 110. For instance, the computed AF indicators 406 are generated when the training ECG segments 404 are input into the analyzer 204. In various implementations, the computed AF indicators 406 indicate whether the classifier 110 has identified AF in the training ECG segments 404. For example, one of the computed AF indicators 406 may indicate that a first training ECG segment 404 does not indicate AF, and another of the computed AF indicators 406 may indicate that a second training ECG segment 404 does indicate AF. In various implementations, the computed AF indicators 406 include one or more misclassifications. For example, the computed AF indicators 406 may incorrectly provide that a third training ECG segment 404 indicates AF and/or incorrectly provide that a fourth training ECG segment 404 does not indicate AF.

In some examples in which the predictive model 304 is configured to exclusively distinguish between false positive and true positive segments evaluated by the classifier 110, the training data 402 may exclusively include training ECG segments 404 that are classified by the classifier 110 as including AF, wherein at least a portion of those training ECG segments 404 actually do not depict AF. In cases in which the predictive model 304 includes a first ML model configured to distinguish between false positive and true positive segments, as well as a second ML model configured to distinguish between false negative and true negative segments, the first ML model may be trained using a different portion of the training data 402 than the second ML model. For instance, the first ML model may be trained using a portion of the training ECG segments 404 that are classified as including AF by the classifier 110, and the second ML model may be trained using a portion of the training ECG segments that are classified as not including AF by the classifier 110. The first ML model may be trained based on both false positive and true positive segments in the training ECG segments 404, and the second ML model may be trained based on both false negative and true negative segments in the training ECG segments 404.

The ground truth AF indicators 408 may include accurate classifications of the training ECG segments 404. In some cases, the ground truth AF indicators 408 are generated by at least one human expert, such as a cardiologist. For example, a human expert may manually review the training ECG segments 404 and label each of them as indicating AF or indicating an absence of AF.

A trainer 410 is configured to train the predictive model 304 using the training data 402. For example, the trainer 410 adjusts and optimizes various parameters 412 within the predictive model 304 based on the training data 402. The trainer 410, for instance, may be implemented by a computing system including one or more processors. The processor(s), for instance, may execute operations of the trainer 410. In some cases, the trainer 410 is part of the analysis system 108 described above with reference to FIG. 1.

In various implementations, the trainer 410 optimizes the parameters 412 using a supervised learning technique. For example, the trainer 410 may generate training accuracy indicators based on mismatches between the computed AF indicators 406 and the ground truth AF indicators 408. In some cases, a first training ECG segment 404 is incorrectly classified by the classifier 110, which can be identified by mismatch between the respective computed AF indicator 406 and the respective ground truth AF indicator 408. The trainer 410, for instance, generates a “false” label for the first training ECG segment 404. A second training ECG segment 404 is correctly classified by the classifier 110, which can be identified by a match between the respective computed AF indicator 406 and the respective ground truth AF indicator 408. In this case, the trainer 410 may generate a “true” label for the second training ECG segment 404.

The representation generator 302, in various cases, generates representations based on the training ECG segments 404. In various implementations, the trainer 410 inputs the representations based on the training ECG segments 404 into the predictive model 304 and adjusts the parameters 412 in order to cause the predictive model 304 to accurately produce the mismatch labels generated based on the computed AF indicators 406 and the ground truth AF indicators 408. For example, the trainer 410 may use backpropagation and/or gradient descent to minimize an error between the mismatch labels and the output of the predictive model 304 when the training ECG segments 404 are input into the predictive model 304. In various cases, once the trainer 410 reduces the error below a threshold level (e.g., less than 1% discrepancy between the mismatch labels and the outputs of the predictive model 304), the predictive model 304 may be defined as “trained.”

After the predictive model 304 is trained, the discriminator 118 may be configured to generate an accuracy indicator 414 based on an unclassified ECG segment 416. In various cases, the unclassified ECG segment 416 is omitted from the training ECG segments 404. The wearable ECG monitor 102, in various cases, may generate ECG data (not illustrated) that includes the unclassified ECG segment 416. For example, the segmenter 202 can be further utilized to generate the unclassified ECG segment 416. In various cases, the unclassified ECG segment 416 is converted into an representation and input into the trained predictive model 304. The predictive model 304 outputs the accuracy indicator 414 based on the representation. The accuracy indicator 414, in various implementations, can be used to determine whether to output a classification of the unclassified ECG segment 416 by the classifier 110.

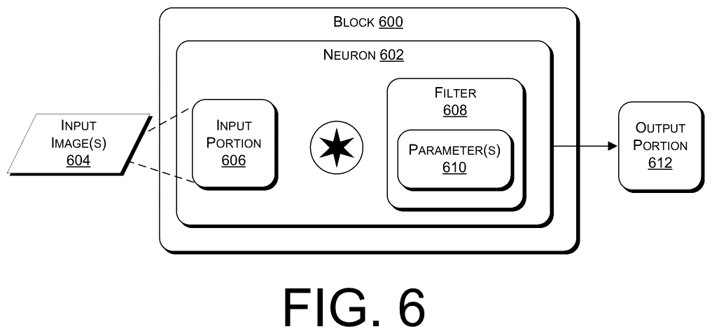

FIG. 5 illustrates example signaling associated with a CNN 502. In various cases, the CNN 502 may be the predictive model 304 described above with reference to FIGS. 3 and 4.

In various implementations, the CNN 502 receives an ECG representation 504 and outputs an accuracy indicator 506 based on the ECG representation 504. The accuracy indicator 506 is a label that provides whether a classifier (e.g., the classifier 110) will accurately classify whether the ECG representation 504 indicates AF (e.g., BAF). For instance, the accuracy indicator 506 could be the accuracy indicator 414 described above with reference to FIG. 4. In various cases, the ECG representation 504 includes an image.

The CNN 502 generates the accuracy indicator 506 using various blocks and/or layers. For instance, the CNN 502 includes first to nth convolutional blocks 508-1 to 508-n, wherein n is a positive integer. The first to nth convolutional blocks 508-1 to 508-n are connected to each other in series. For example, if n is greater than 1, the output of the first convolutional block 508-1 is input into a second convolutional block among the n convolutional blocks 508-1 to 508-n. Each of the first to nth convolutional blocks 508-1 to 508-n includes one or more convolutional layers configured to cross-correlate and/or convolve a filter (e.g., an image filter) with an input (e.g., at least a portion of an input image). In various implementations, parameters that define the filter are optimized during training of the CNN 502. In some cases, individual convolutional blocks 508-1 to 508-n further include a normalization layer and/or an activation layer. For instance, each of the convolutional blocks 508-1 to 508-n includes a batch normalization layer and a Rectified Linear Unit (ReLU) activation layer.

The CNN 502, in various cases, further includes an activation block 510 in series with the nth convolutional block 508-n. The activation block 510, for instance, applies an activation function to the output of the nth convolutional block 508-n. The activation function, in some cases, is a softmax, sigmoid, or argmax function. In various implementations, the activation block 510 outputs the accuracy indicator 506.

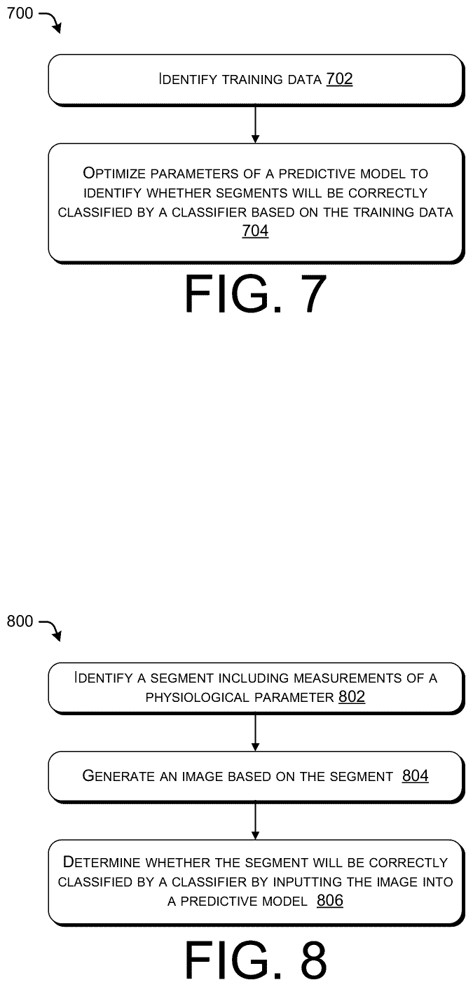

FIG. 6 illustrates an example of a convolutional block 600 in a neural network. In some examples, the block 600 can represent any of the convolutional blocks and/or layers described herein.

The convolutional block 600 may include multiple neurons, such as neuron 602. In some cases, the number of neurons may correspond to the number of pixels in at least one input image 604 input into the block 600. Although one neuron is illustrated in each of FIG. 6, in various implementations, block 600 can include multiple rows and columns of neurons.

In particular examples, the number of neurons in the block 600 may be less than or equal to the number of pixels in the input image(s) 604. In some cases, the number of neurons in the block 600 may correspond to a “stride” of neurons in the block 600. In some examples in which first and second neurons are neighbors in the block 600, the stride may refer to a lateral difference in an input of the first neuron and an input of the second neuron. For example, a stride of one pixel may indicate that the lateral difference, in the input image(s) 604, of the input of the first neuron and the input of the second neuron is one pixel.

Neuron 602 may accept an input portion 606. The input portion 606 may include one or more pixels in the input image(s) 604. A size of the input portion 606 may correspond to a receptive field of the neuron 602. For example, if the receptive field of the neuron 602 is a 3×3 pixel area, the input portion 606 may include at least one pixel in a 3×3 pixel area of the input image(s) 604. The number of pixels in the receptive field that are included in the input portion 606 may depend on a dilation rate of the neuron 602.

In various implementations, the neuron 602 may convolve (or cross-correlate) the input portion 606 with a filter 608. The filter may correspond to at least one parameter 610, which may represent various optimized numbers and/or values associated with the neuron 602. In some examples, the parameter(s) 610 are set during training of a neural network including the block 600.

The result of the convolution (or cross-correlation) performed by the neuron 602 may be output as an output portion 612. In some cases, the output portion 612 of the neuron 602 is further combined with outputs of other neurons in the block 600. The combination of the outputs may, in some cases, correspond to an output of the block 600. Although FIG. 6 depicts a single neuron 602, in various examples described herein, the block 600 may include a plurality of neurons performing operations similar to the neuron 602. In addition, although the convolutional block 600 in FIG. 6 is depicted in two dimensions, in various implementations described herein, the convolutional block 600 may operate in three dimensions.

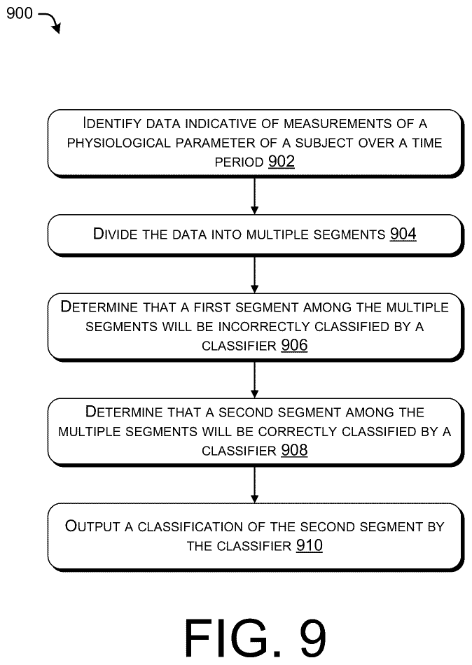

FIG. 7 illustrates an example process 700 for training a discriminator to identify whether a classifier will accurately classify input data. The process 700 may be implemented by an entity including a computing system, at least one processor, the analysis system 108, the trainer 410, or any combination thereof.

At 702, the entity identifies training data. In various cases, the training data may include multiple segments of physiological parameter data. Each segment, for instance, includes measurements of a physiological parameter. In various cases, the training data includes segments that are obtained from multiple subjects in a population. The segments include one or more first segments that are indicative of a condition and one or more second segments that are indicative of the absence of the condition. In various cases, one or more of the segments include artifact and/or noise. For instance, one or more of the segments include a motion artifact. In some implementations, the segments are converted to images, such as spectrograms or other images indicative of the segments.

The training data, in various implementations, further includes classifier-generated indicators of whether the condition is indicated in the segments. For example, the classifier is configured to identify one or more features that are indicative of the condition in each individual segment. However, the classifier may misidentify the condition in one or more of the segments. The training data may further include ground truth indicators, which provide whether the condition is actually present in the various segments. In some examples, the ground truth indicators are generated by an expert grader, such as a physician. In some cases, the training data includes mismatches of the classifier-generated indicators and the ground truth indicators. For instance, the training data may indicate whether the classifier-generated indicators are correct or erroneous.

At 704, the entity optimizes, based on the training data, parameters of a predictive model to identify whether segments will be correctly classified by a classifier. The predictive model may include at least one ML model, such as at least one CNN. The parameters, for instance, include values of kernels within one or more convolutional layers of the CNN. In various cases, the entity modifies the parameters of the predictive model until the predictive model accurately outputs the mismatches in response to receiving the segments and/or images as inputs. The entity, in various implementations, utilizes a supervised learning process to train the predictive model.

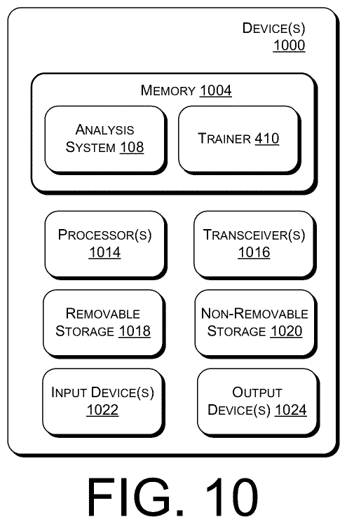

FIG. 8 illustrates an example process 800 for determining whether a classifier will accurately classify input data. The process 800 may be implemented by an entity including a computing system, at least one processor, the analysis system 108, the discriminator 118, or any combination thereof.

At 802, the entity identifies a segment including measurements of a physiological parameter. In various implementations, the measurements may be generated by a wearable device. In some cases, the segment is a portion of a longer data set including measurements taken over an extended time period, such as a time period that is greater than 1 minute, 30 minutes, or one hour. The entity may, in some cases, identify the segment by dividing the longer data set.

At 804, the entity generates an image based on the segment. In various implementations, the entity generates a spectrogram of the segment.

At 806, the entity determines whether the segment will be correctly classified by a classifier by inputting the image into a predictive model. The predictive model may include one or more ML models, such as one or more CNNs. The predictive model, for instance, has been previously trained based on training data obtained from a population. In some cases, the segment includes measurements from a subject that is omitted from the population. In various implementations, the predictive model may output an indication of whether the classifier will generate a correct classification of the segment or an incorrect classification of the segment.

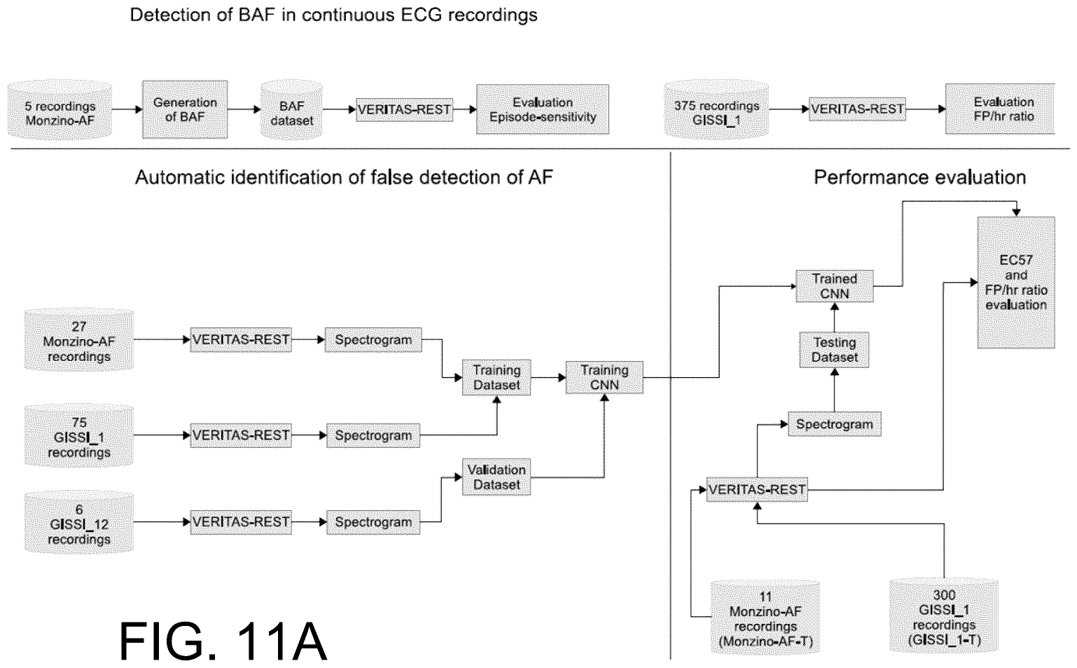

FIG. 9 illustrates an example process 900 for classifying whether input data is indicative of a condition. The process 900 may be implemented by an entity including a computing system, at least one processor, the analysis system 108, the classifier 110, the discriminator 118, or any combination thereof.

At 902, the entity identifies data indicative of measurements of a physiological parameter of a subject over a time period. In some cases, the entity receives a signal indicating the data from an external device (e.g., a wearable monitor). In some examples, the entity itself generates the data by detecting the physiological parameter. The time period, for instance, may be greater than 1 minute, 30 minutes, or one hour.

At 904, the entity divides the data into multiple segments. In various implementations, the segments are overlapping and/or nonoverlapping. In some examples, each segment represents measurements taken over a duration that is significantly shorter than the time period. For instance, the duration may be 10 seconds, 30 seconds, 1 minute, five minutes, or ten minutes.

At 906, the entity determines that a first segment among the multiple segments will be incorrectly classified by a classifier. In some cases, the first segment includes an artifact that will result in an erroneous classification by the classifier. For instance, the first segment may include a motion artifact. In various implementations, the entity generates a first image based on the first segment. In some cases, the first image is a spectrogram of the first segment. The entity may further input the first image into a predictive model. The predictive model, for instance, includes one or more ML models. In some cases, the predictive model includes a CNN. The predictive model may be previously trained. In various implementations, the predictive model outputs an indication that the classifier will incorrectly classify the segment based on receiving the first image as an input.

At 908, the entity determines that a second segment among the multiple segments will be correctly classified by the classifier. For example, the second segment may omit a significant artifact. In various cases, the entity generates a second image based on the second segment. In some cases, the second image is a spectrogram of the second segment. The entity may input the second image into the predictive model. The predictive model may output an indication that the classifier will correctly classify the segment based on receiving the second image as an input.

At 910, the entity outputs a classification of the second segment by the classifier. In some implementations, the entity inputs the second segment into the classifier, and receives the classification as an output of the classifier. According to some cases, the entity refrains from inputting the second segment into the classifier, which can reduce a computing burden of the entity. In various implementations, the entity transmits a signal indicating the classification. In some cases, the entity outputs the second segment. In some implementations, the entity presents the classification and/or the second segment to a user, such as a clinician.

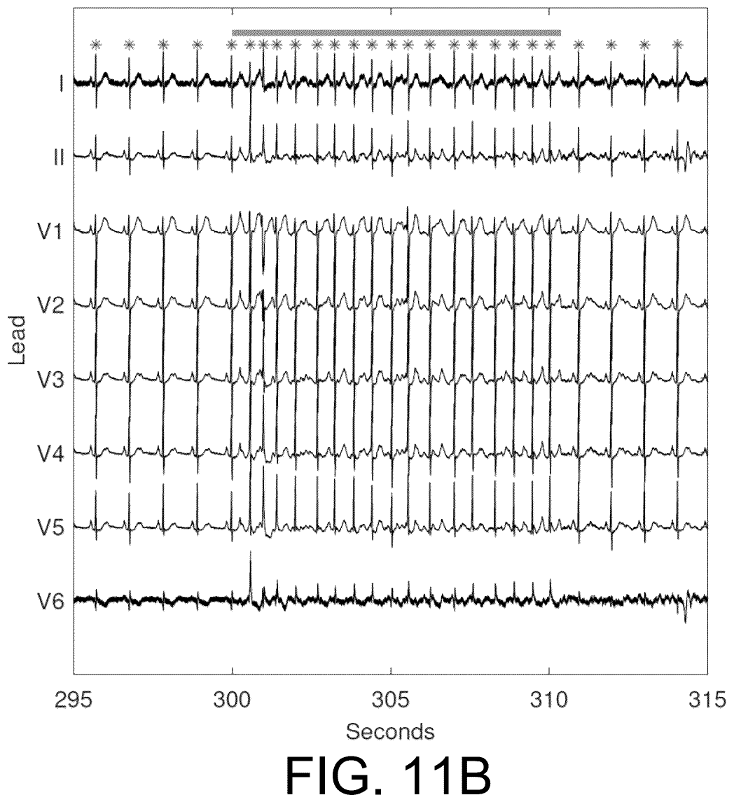

FIG. 10 illustrates at least one example device 1000 configured to enable and/or perform the some or all of the functionality discussed herein. Further, the device(s) 1000 can be implemented as one or more server computers, a network element on a dedicated hardware, as a software instance running on a dedicated hardware, or as a virtualized function instantiated on an appropriate platform, such as a cloud infrastructure, and the like. It is to be understood in the context of this disclosure that the device(s) 1000 can be implemented as a single device or as a plurality of devices with components and data distributed among them.

As illustrated, the device(s) 1000 comprise a memory 1004. In various embodiments, the memory 1004 is volatile (including a component such as Random Access Memory (RAM)), non-volatile (including a component such as Read Only Memory (ROM), flash memory, etc.) or some combination of the two.

The memory 1004 may include various components, such as the analysis system 108 and/or trainer 410. The analysis system 108 and/or trainer 410 may include models, methods, threads, processes, applications, or any other sort of executable instructions. The analysis system 108 and/or trainer 410 stored in the memory 1004 can also include files and databases.

The memory 1004 may include various instructions (e.g., instructions in the analysis system 108 and/or trainer 410), which can be executed by at least one processor 1014 to perform operations. In some embodiments, the processor(s) 1014 includes a Central Processing Unit (CPU), a Graphics Processing Unit (GPU), or both CPU and GPU, or other processing unit or component known in the art.

The device(s) 1000 can also include additional data storage devices (removable and/or non-removable) such as, for example, magnetic disks, optical disks, or tape. Such additional storage is illustrated in FIG. 10 by removable storage 1018 and non-removable storage 1020. Tangible computer-readable media can include volatile and nonvolatile, removable and non-removable media implemented in any method or technology for storage of information, such as computer readable instructions, data structures, program modules, or other data. The memory 1004, removable storage 1018, and non-removable storage 1020 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 1000. Any such tangible computer-readable media can be part of the device(s) 1000.

The device(s) 1000 also can include input device(s) 1022, such as a keypad, a cursor control, a touch-sensitive display, voice input device, etc., and output device(s) 1024 such as a display, speakers, printers, etc. In some implementations, the input device(s) 1022 include one or more sensors configured to detect one or more physiological parameters of a subject. In various cases, the device(s) 1000 include one or more wearable devices. In particular implementations, a user can provide input to the device(s) 1000 via a user interface associated with the input device(s) 1022 and/or the output device(s) 1024.

As illustrated in FIG. 10, the device(s) 1000 can also include one or more wired or wireless transceiver(s) 1016. For example, the transceiver(s) 1016 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, the transceiver(s) 1016 can utilize Multiple-Input/Multiple-Output (MIMO) technology. The transceiver(s) 1016 can include any sort of wireless transceivers capable of engaging in wireless, Radio Frequency (RF) communication. The transceiver(s) 1016 can also include other wireless modems, such as a modem for engaging in Wi-Fi, WiMAX, Bluetooth, or infrared communication.

In some implementations, the transceiver(s) 1016 can be used to communicate between various functions, components, modules, or the like, that are comprised in the device(s) 1000.

### EXPERIMENTAL EXAMPLE

The present experimental example describes the feasibility of detecting BAF episodes in continuous ECG recordings using a commercial algorithm intended for analysis of ECG recordings acquired at rest. Various implementations of the present disclosure utilize multiple leads to detect BAF and other conditions. One option for detecting BAF episodes in continuous ECG recordings using multi-lead information would be to analyze the signals with a commercial algorithm intended for analysis of 10-second ECG signals acquired at rest (referred to herein as a “resting ECG algorithm”). Commercial algorithms are governed by international regulations like those from the European Medical Device Regulation, the U.S. Food & Drug Administration, and the China Food & Drug Administration. A 12-lead resting ECG is by far the most used ECG examination. Additionally, cleared commercial resting ECG algorithms are developed using large resting ECG databases with a wide variety of arrhythmias and other cardiac pathologies that, in principle, could also be detected by such algorithms on continuous ECG recordings. Resting ECG algorithms are expected to be more sensitive to detect BAF than algorithms for the analysis of continuous ECG recordings as they provide an interpretation from ECG-signals of only 10 seconds.

Nevertheless, various resting ECG algorithms are not designed to deal with excessive motion artifact, and consequently they may produce many false detections (FD) when analyzing continuous ECG recordings acquired with Holter recorders or telemetry systems. This experimental example utilizes a resting ECG algorithm to detect BAF in a continuous ECG recording by reducing the impact of motion artifact and other noise in the continuous ECG recording.

Time-frequency analysis facilitates identification of such artifacts in continuous ECG recordings. The spectrogram and the scalogram provide a signal representation in the time-frequency domain, the former results from the short-time Fourier transform and the latter from the continuous wavelet transform.

In this Experimental Example, detection performance was measured with respect to episode sensitivity and FD rate. A further objective of this example was to investigate the feasibility of using a CNN and spectrogram images to reduce the number of FDs by the resting ECG algorithm resulting from motion artifact usually present in continuous ECG recordings. Finally, telemetry and Holter ECG recordings were used to compare the performance of the proposed approach to the performance of a commercial algorithm for the diagnosis of cardiac arrhythmia in continuous ECG recordings following the recommendations in the American National Standard EC57 for testing and reporting performance results of cardiac rhythm and ST segment measurement algorithms.

### Materials and Methods

### Databases

The proprietary Monzino database was collected in the Ventricular Intensive Care unit for intensive treatment of ventricular arrhythmias at the Cardiologic Monzino Center (Milan, Italy), following the declaration of Helsinki and local ethical regulations. The database includes 5009 ECG telemetry recordings that consist of consecutive continuous recordings from 861 consecutive patients. The recordings were acquired with the Mortara Intrument X12+ Digital Telemetry and Surveyor Central Station™ from hospitalized adult patients with different arrhythmias. The recordings have 12 leads sampled at 500 Hz with 2.5 μV per least significant bit resolution.

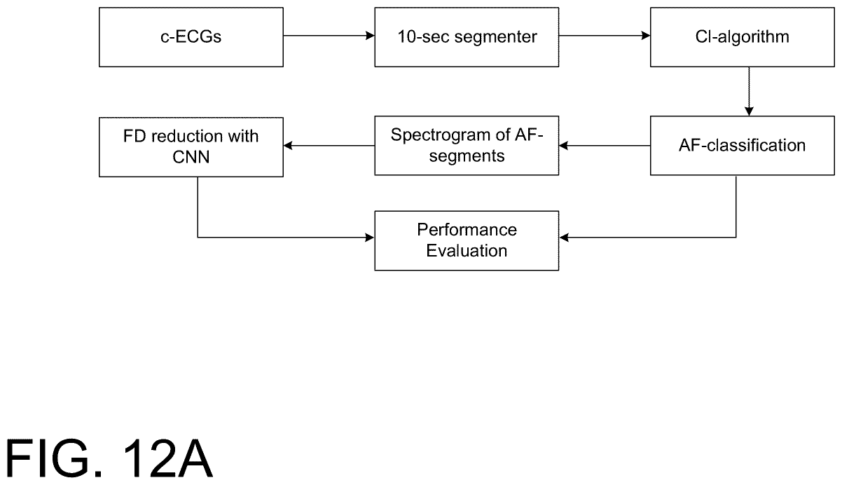

FIG. 11A illustrates an example schematic indicating how databases were used in the development and testing of the proposed AF detector. The Monzino-AF database is a subset of the Monzino database which includes 38 ECG excerpts from 35 recordings, all manually reviewed by experienced ECG-readers and confirmed by one cardiologist. The recordings in the Monzino-AF have duration of 1.58±0.73 hours (mean±std), and the total duration in the database is 61.56 hours. Hereinafter, the excerpts in Monzino-AF will be referred to as recordings. The recordings were manually annotated for AF and atrial flutter (aflutter) by qualified ECG-readers and confirmed by one cardiologist. One recording has 8 episodes of BAF or aflutter and 1 AF episode longer than 30-second, 25 recordings have only 1 AF episode longer than 30-second, 3 recordings have only 1 aflutter episode longer than 30-second, and 9 recordings do not have episodes of AF or aflutter. Aflutter was included with AF because the clinical imperative for aflutter can be very similar to AF. Hereinafter, AF is used to indicate both atrial fibrillation and atrial flutter in the Monzino-AF database. The 8 BAF episodes have duration 7.44±2.95 seconds (mean±std), and the duration of the AF episodes longer than 30-second in the 29 recordings is 1.55±0.63 hours (mean±std). The total AF duration in the database is 44.94 hours. The Monzino-AF is used herein to evaluate the sensitivity of BAF detection, and to generate spectrogram images from ECG segments with AF for the training process of the CNN; see FIG. 11A.

The GISSI-HF study is a randomized, large-scale, double-blind, placebo-controlled study to assess two pharmacological agents in symptomatic heart failure patients. The study was performed following the declaration of Helsinki and approved by the local Ethics Committees. Before enrollment, all patients provided written informed consent. Eligible patients were men and women aged 18 years or older, with diagnosed heart failure of any cause. The design of the study included a Holter substudy to assess Holter-derived autonomic variables. In this substudy, 388 patients in sinus rhythm underwent 24-hour Holter recording at the time of enrollment and at 3 and 12 months after randomization. Patients performed Holter cardiac monitoring using the Mortara Instrument H12+ Digital Holter Recorder™ acquiring 12 leads sampled at 1000 Hz with 2.5 μV per least significant bit resolution.

Since the presence of clinically confirmed AF was an exclusion criteria for enrollment in the GISSI-HF study, little or no AF was expected in the recordings from the first visit. However, some AF episodes were expected in the recordings from the third visit, since patients have a relative high risk to develop AF over time due to age and heart failure history. After these assumptions, this experimental example used data from the first and third visit. For the first visit, there were 388 recordings acquired from patients in SR. 13 recordings were excluded due to missing data. Hereinafter, the remaining 375 recordings from the first visit will be referred to as GISSI 1. The recordings in the GISSI 1 have a duration 23.95±1.03 hours (meantstd). The GISSI 1 is used to evaluate ratio of FD of AF, and to generate spectrogram images from ECG segments without AF for the training process of the CNN; see FIG. 11A. For the third visit, there were 372 recordings acquired. These recordings were analyzed with the VERITAS Arrhythmia/ST AnalysisTM, and AF was detected in 182 recordings. Detections of AF from 20 recordings with the highest AF burden were manually reviewed by qualified ECG-readers, confirming AF only in 3 recordings. The 3 recordings with confirmed AF and 3 recordings with misdetected AF will be referred to as GISSI 12. These 6 recordings have a duration of 24 hours each. The duration of the AF episodes is 1.00±4.69 hours (meantstd), and the total AF duration is 26.08 hours. The GISSI 12 is used generate spectrogram images from ECG segments with and without AF for the training process of the CNN; see FIG. 11A.

| Database | Monzino-AF | GISSI_1 | GISSI_12 |
|---|---|---|---|
| Recordings | 38 | 375 | 6 |
| Total Duration | 62 hours | 8981 hours | 144 hours |
| of recordings | | | |
| Total AF duration | 45 hours | 0 hours | 26 hours |
| Leads | 12 | 12 | 12 |
| Used for training CNN | Yes | Yes | No |
| Used for validating CNN | No | No | Yes |
| Used for testing CNN | Yes | Yes | No |

Table 1 summarizes the databases and the box diagram in FIG. 11A provides an outlook on how databases were used in this study.

### Detection of BAF in Continuous ECG Recordings

The VERITAS Resting ECG Interpretation™ (VERITAS-REST) is an algorithm designed for processing and analyzing 10-second resting ECG recordings. In this study, the VERITAS-REST version 7.5.0 was used for analysis. The input to this algorithm is 10-second ECG recording sampled either at 500 or 1000 Hz including 8 independent leads (standard leads I, II, V1-V6) with precordial leads referenced to the Wilson terminal. The algorithm outputs a beat list (list of detected beats and classifications), various measurements (global and lead measurements), and various types of interpretation statements, including a single rhythm interpretation statement.

To analyze continuous ECG recordings using a resting ECG algorithm, the continuous ECG recordings were divided into 10-second ECG segments using a 5-second sliding window. Then, each 10-second ECG segment was analyzed with the VERITAS-REST for which the algorithm produced one rhythm statement for the segment. Finally, statements from all segments were used to generate a sample-by-sample labeling for AF. For this purpose, all samples in a 10-second ECG segment producing a rhythm statement of AF or aflutter were labeled as AF, all other rhythm statements were labeled as non-AF.

To investigate the feasibility of using VERITAS-REST for BAF detection in continuous ECG recordings, ECG recordings were generated with BAF episodes of variable length through shortening longer AF episodes to 3, 5, 10, 15, 20, and 30 seconds from recordings in the Monzino-AF database. Similarly, AF episodes of length 60, 90, 120, 150, and 180 seconds were also generated. The recordings were generated from five recordings having a single AF episode preceded and followed by at least 5 minutes of SR. To generate the recordings, the AF episode was truncated to a specific duration and concatenated with the SR segment after AF, so that AF onsets in the dataset were defined as physiological onsets, whereas AF offsets were artificially generated. The AF episode was truncated at half the distance between the R-peak of the last beat before the set AF duration and the R-peak of the next beat. The start of the following SR segment was halfway between the R-peak of the last physiological AF beat and the R-peak of the first sinus beat.

FIG. 11B illustrates an example of a generated ECG recording showing the onset and offset of a 10-second AF episode. Red asterisks represent beat locations and the green bar highlights the AF episode.

The generated recordings were used to investigate detection sensitivity for BAF in continuous ECG recordings with the VERITAS-REST, and the performance was evaluated in terms of episode-sensitivity. Further, the GISSI 1 was analyzed with the VERITAS-REST to investigate the ratio of FD of AF per hour, see supra.

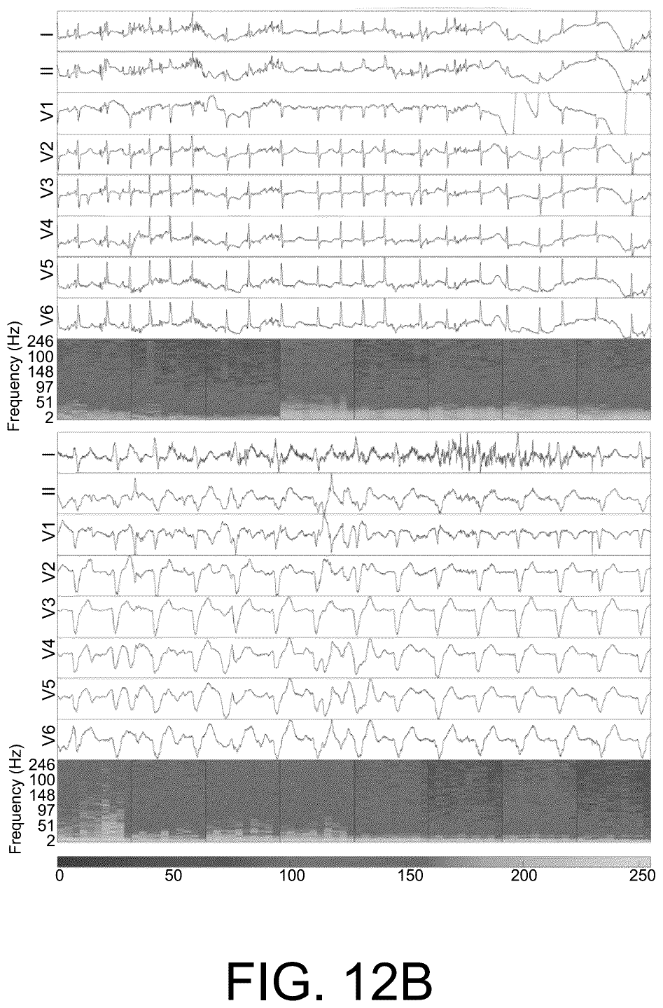

FIG. 12A illustrates an example schematic indicating how ECGs are classified as AF or non-AF by a computerized resting ECG algorithm (CI-algorithm). In this example, a continuous ECG (c-ECG) is divided into 10-second segments that are classified by the CI-algorithm as atrial fibrillation (AF) or non-AF. Spectrograms generated from segments classified as AF are fed to a CNN trained to identify false detections (FD). Classified segments are unified into a sample-by-sample label for the c-ECG and used to evaluate episode and duration performance and to count the number of false AF alarms per hour (FA/hr).

To identify 10-second ECG segments that are incorrectly classified as AF or aflutter by the VERITAS-REST, a CNN was trained with spectrogram images generated from 10-second ECG segments. For the training, the 10-second ECG segments were labeled as true AF (TAF) if correctly classified as AF by the VERITAS-REST and labeled as false AF (FAF) if incorrectly classified as AF by the VERITAS-REST. To generate spectrogram images, the ECG segments were resampled to 500 Hz and the leads were concatenated one after the other resulting in a signal of 40000 samples (5000 samples each lead). The spectrogram images of the resulting signal was generated using the short-time Fourier-transform with non-overlapping windows corresponding to 1-second using 512-point fast Fourier transform. The spectrogram images were generated and saved to PNG format using MATLAB (The Math Works, 2021a). Each of the resulting spectrogram images had dimensions of 80×257 pixels.

FIG. 12B illustrates (top) a 10-second ECG segment correctly labeled as AF by the Cl-algorithm and the CNN, and (bottom) a 10-second ECG segment incorrectly labeled as AF by the Cl-algorithm identified as a false detection by the CNN. Spectrograms generated according to the methods described above are shown below the corresponding ECG traces.

| | Kernel size | Stride | |
|---|---|---|---|
| Layer | (H, L, W) | (H, L) | Activation |
| Input | — | — | 257 × 80 × 1 |
| Convolutional 1 | (3, 10, 10) | (1, 10) | 257 × 8 × 10 |
| Batch Normalization 1 | — | — | 257 × 8 × 10 |
| ReLu activation 1 | — | — | 257 × 8 × 10 |
| Max Pooling 1 | (3, 3, 10) | (1, 5) | 257 × 2 × 10 |
| Convolutional 2 | (3, 10, 10) | (1, 10) | 257 × 1 × 10 |
| Batch Normalization 2 | — | — | 257 × 1 × 10 |
| ReLu activation 2 | — | — | 257 × 1 × 10 |
| Max Pooling 2 | (3, 3, 10) | (2, 2) | 129 × 1 × 10 |
| Convolutional 3 | (2, 4, 20) | (2, 2) | 65 × 1 × 20 |
| Batch Normalization 3 | — | — | 65 × 1 × 20 |
| ReLu activation 3 | — | — | 65 × 1 × 20 |
| Fully Connected | — | — | 1 × 1 × 2 |
| Softmax | — | — | 1 × 1 × 2 |
| Output | — | — | 1 × 1 × 2 |

Table 2 indicates properties of the CNN. Letters H, L, and W stand for height, length, and width, respectively. Zero padding was used on the edges on the layers.

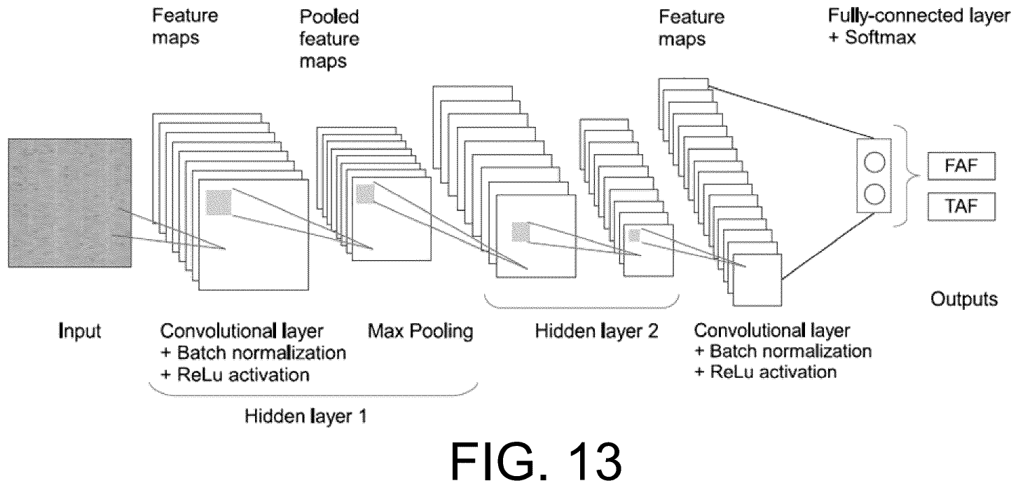

FIG. 13 illustrates the architecture of the CNN used in the study. Hidden layer 2 contains the same set of elements as hidden layer 1. The architecture of the CNN has 3 convolutional layers, 3 batch normalization layers, 3 ReLu activation layers, 2 max pooling layers, 1 fully connected layer, 1 softmax layer, and 1 classification layer. This architecture includes 7622 learnable parameters, and is summarized in FIG. 13 and Table 2. The settings for training include using the stochastic gradient descent with momentum minimizing the binary cross-entropy function as cost function, learning rate of 0.01, L2 regularization factor of 0.0001, momentum contribution of 0.9, and batches of 500 spectrogram images randomly shuffled every epoch. The CNN was trained using the function trainNetwork from the Deep Learning Toolbox from MATLAB (The Math Works, 2021a). The CNN was trained to perform the classification between TAF images and FAF images.

The process for training a CNN can involve two stages: validation and testing. The former is normally used to find the best architecture and settings of the CNN to accomplish a specific task while the latter is used to evaluate its performance. It is common to use the same training dataset for both stages, however the validation and testing datasets can be mutually exclusive to prevent biasing its performance on the testing dataset. In this example, spectrogram images were first generated from the different databases and then split them into three mutually exclusive datasets: training, validation, and testing. Monzino-AF and GISSI 1 were used to generate training and testing datasets, and GISSI 12 to generate the validation dataset, see FIG. 11A. 29802 spectrogram images were labeled as TAF. from Monzino-AF recordings, 313 spectrogram images were labeled as FAF from Monzino-AF recordings, and 131997 spectrogram images were labeled as FAF from the GISSI 1 recordings were produced. A randomly selected assortment of the TAF spectrogram images generated from 80% of the Monzino-AF database (23 recordings) were used in the training stage, and the images from the remaining 20%(6 recordings) were left for testing. The recording with 8 episodes of BAF was used in the testing dataset. The FAF from 4 Monzino-AF recordings randomly selected were used in the training and the FAF images from the remaining recordings for testing. The spectrogram images from 75 recordings that were randomly selected from the GISSI 1 were used in the training stage, and for the testing stage, the images resulting from the remaining 300 recordings were included. During the random selection of the recordings, training datasets were constrained to be balanced to avoid biasing the learning process of the CNN. Hereinafter, the testing subsets from each database are referred to as Monzino-AF-T and GISSI 1-T, respectively. Additionally, 17191 TAF images and 6811 FAF images were produced from the GISSI 12 recordings. The validation dataset included the images from the GISSI 12. The training set was generated 10 times and validated on the data set from the GISSI 12.

Standard classification metrics were used to evaluate the ability of the CNN to classify the spectrogram images between FAF and TAF. The performance of the CNN indicates how accurately the network identifies FAF images from TAF. For instance, the network is designed to identify the ECG segments that are incorrectly classified as AF by the VERITAS-REST. Therefore, the classification ability of the CNN can be evaluated by determining the number of images correctly classified as FAF (true positives, TP), the number of images correctly classified as TAF (true negatives, TN), the number of images falsely classified as FAF (false positives, FP), and the number of images falsely classified as TAF (false negatives, FN). From these counts it is possible to calculate the accuracy (Acc), sensitivity (Se), specificity (Sp), positive predictive value (PPV) of the CNN. These metrics are defined in equations (1)-(4) taking values in the interval [0,1], where 1 corresponds to perfect performance.

$$Acc = \frac{TP + TN}{TP + TN + FP + FN} , \qquad ( 1 )$$

$$Se = \frac{TP}{TP + FN} , \qquad ( 2 )$$

$$Sp = \frac{TN}{TN + FP} , \qquad ( 3 )$$

$$PPV = \frac{TP}{TP + FP} , \qquad ( 4 )$$

### Electrocardiomatrix for Detecting Atrial Fibrillation

The electrocardiomatrix (ECM) is a technique proposed by Li et al (2015), J Integr Cardiol 1, 124-128, to simplify manual inspection of ECG recordings. Lee et al. (2018), Journal of electrocardiology 51, S121-S125 showed that the ECM is a reliable method for accurate detection of AF when applied to long-term ECG recordings for subsequent analysis by experienced ECG-readers. The ECM technique was later used by Salinas-Martinez et al. (2021), Frontiers in Physiology 12, 1-16, to train a CNN for automatic identification of BAF episodes based on ECM-images generated from ECG segments containing 10-beats plus 2.5 seconds after the last beat and 0.5 seconds before the first beat. The ECM-image was constructed by dividing an ECG segment into ten, partially overlapping, subsegments of 3.0 seconds for which the ith subsegment starts 0.5 seconds before the R peak of the ith beat in the segment, 1≤i≤10. Further, the ten subsegments were decimated and aligned vertically to the first R peak in each subsegment, and then saved as an intensity image, referred to as the ECM-image. The CNN was trained with 1.6811×106 ECM-images from AF and non-AF segments, and tested on the MIT-BIH Atrial Fibrillation database (AFDB) (Goldberger et al. (2000, Circulation 101, e215-e220), MIT-BIH Arrhythmia database (Moody and Mark (2001), IEEE Engineering n Medicine and Biology Magazine 20, 45-50), and the Monzino-AF database, which were not used for training. In this disclosure, the approach by Salinas-Martinez et al. Frontiers in Physiology 12, 1-16. (2021) was evaluated on the Monzino-AF-T and the GISSI 1-T for comparison.

### Performance Evaluation

For performance evaluation, the recordings from the Monzino-AF-T and from the GISSI 1-T that were used in the testing stage of the CNN were utilized. It is worth recalling that the spectrogram images are generated from ECG segments classified as AF or aflutter by the VERITAS-REST, and that the CNN can be intended to reduce the number of FD. Therefore, the AF detections made by the VERITAS-REST on continuous ECG recordings were updated based on the outputs of the CNN, hereinafter VERITAS-REST+CNN. The AF detections by the VERITAS-REST+CNN are mapped to a sample-by-sample labeling, and then compared to the performance of the VERITAS-REST. Performance was measured following the recommendations in the EC57 standard.

The EC57 standard provides a guideline for testing and reporting performance of algorithms for cardiac rhythm and has been recognized as a consensus standard for medical devices by the U.S. Food & Drug Administration. This standard is also recognized by the international standard IEC60601-2-47:2015 for the basic safety and essential performances of ambulatory electrocardiograms. The EC57 standard emphasizes the importance of reporting statistics for the number of episodes detected as well as for the duration of the episodes. The metrics suggested by the standard to evaluate the performance of an algorithm are: episode sensitivity (Se_{Epi}), episode positive predictive value (PPV_{Epi}), duration sensitivity (Se_{Dur}), and duration positive predictive value (PPV_{Dur}). These metrics are defined as follows:

$${Se}_{Epi} = \frac{{TP}_{Epi}}{{TP}_{Epi} + {FN}_{Epi}} , \qquad ( 5 )$$

$${PPV}_{Epi} = \frac{{TP}_{Epi}}{{TP}_{Epi} + {FP}_{Epi}} , \qquad ( 6 )$$

$${Se}_{Dur} = \frac{{T}_{AF} ⋂ {\hat{T}}_{AF}}{{T}_{AF}} , \qquad ( 7 )$$

$${PPV}_{Dur} = \frac{{T}_{AF} ⋂ {\hat{T}}_{AF}}{{\hat{T}}_{AF}} , \qquad ( 8 )$$

where TP_{Epi} is the number of correctly detected AF episodes, FN_{Epi} is the number of undetected AF episodes, FP_{Epi} is the number of incorrectly detected AF episodes, TAF is the total duration of AF manually annotated, and T\hat{ }AF is the total duration of AF detected by the algorithm. Additionally to these metrics, the ratio FD/hr was included to investigate the number of false detections per hour when analyzing continuous ECG recordings. These metrics were used first to evaluate the performance of the algorithms to detect BAF episodes on the generated ECG recordings, and later for the detection of AF in the Monzino-AF-T and GISSI 1-T databases.

### Results

### Detection of BAF

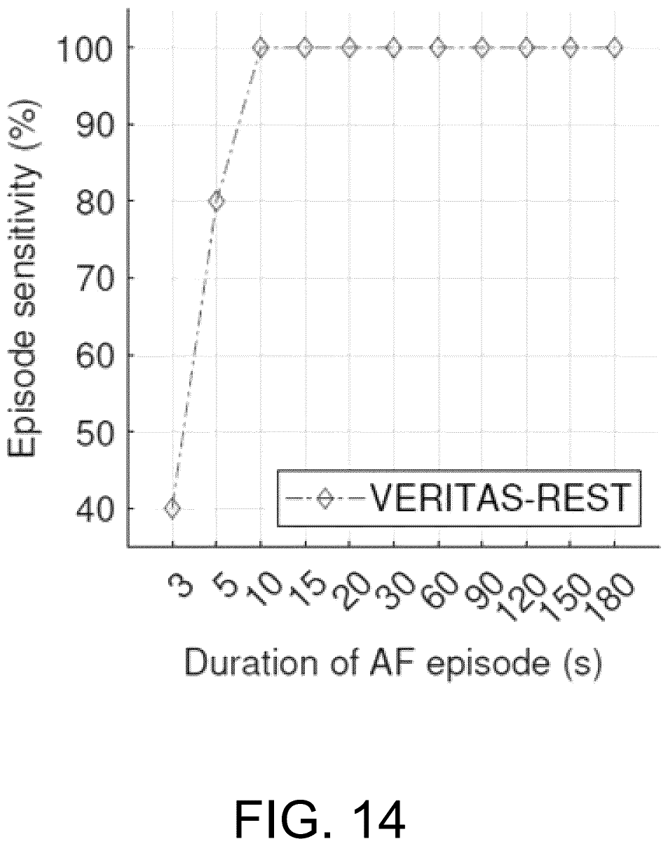

FIG. 14 illustrates an example of episode sensitivity for BAF detection of the VERITAS-ARRH and the VERITAS-REST on the generated ECG recordings with different duration of AF.

The recordings generated using the techniques described herein were analyzed with the VERITAS-REST algorithms for detecting the BAF episodes of variable length. The goal was to detect the BAF episodes, hence performance was evaluated in terms of Se_{Epi} and summarized in FIG. 14. In this experimental example, there was a progressive improvement for the VERITAS-REST as the AF episode duration increased, achieving 100% sensitivity on AF episode longer than 10 seconds. These results suggest that the VERITAS-REST could be used for detecting AF episodes in continuous ECG recordings. The number of FD in continuous ECG recordings was also investigated by running the VERITAS-REST on the GISSI 1, as it was collected from patients in SR, producing 4.26 FD/hr.

### Identification of Falsely Detected AF Episodes

| | Monzino-AF | GISSI_1 |
|---|---|---|
| AF | 21124 | 0 |
| False Detection | 66 | 22770 |

Table 3 provides the datasets of 10-second segments classified as AF by VERITAS-REST used for training.

To reduce the number of FD/hr produce by the VERITAS-REST, a CNN was trained to identify ECG segments incorrectly labeled as AF. Spectrogram images from a subset of 21124 segments that were correctly labeled as AF by VERITAS-REST and 22836 that were incorrectly labeled as AF by VERITAS-REST are used to train the CNN to identify FAF segments (Table 3). The remaining 8678 TAF and 92293 FAF spectrogram images not used for training were used to test the CNN. The CNN was first validated using the GISSI 12. In this experimental example, during the validation stage, the CNN classified the images with Acc=72.81±7.81, Se=80.24±5.76, PPV=52.73±8.62, and Sp=69.87±11.25. The average time needed for training is 276.51±8.74 seconds. For testing purposes, the CNN reaching the highest Acc performance on the validation set was used and tested on the Monzino-AF-T and the GISSI 1-T datasets.

| | | Monzino-AF | | GISSI_1 |
|---|---|---|---|---|
| VERITAS- | Non-AF | 3098 | 67 | 4835348 |
| REST | AF | 257 | 8678 | 92046 |
| Output | | Non-AF | AF | Non-AF |

Target

Table 4 Provides the VERITAS-REST Classification Performance of the 10-Second Segments in the Testing Dataset.

| | | Monzino-AF | | GISSI_1 |
|---|---|---|---|---|
| CNN- | Non-AF | 33 | 504 | 82011 |
| Output | AF | 224 | 8174 | 10035 |
| | | Non-AF | AF | Non-AF |

Target

Table 5 provides the CNN classification of the 10-second segments in the testing dataset labeled as AF by VERITAS-REST.

| | | Monzino-AF | | GISSI_1 |
|---|---|---|---|---|
| VERITAS- | Non-AF | 3131 | 571 | 4917359 |
| REST + | AF | 224 | 8174 | 10035 |
| CNN | | Non-AF | AF | Non-AF |

| | |
|---|---|
| Output | Target |

Table 6 provides the combined VERITAS-REST+CNN classification performance of the 10-second segments in the testing dataset.

Classification on the testing datasets produced Acc=89.34%, Se=94.19%, and Sp=88.88, indicating that the CNN is able to identify ECG segments that are incorrectly classified as AF by the VERITAS-REST (see Table 5). The time needed for testing is 25.84 seconds.

### Performance Evaluation

| | VERITAS- | VERITAS- | |
|---|---|---|---|
| | REST | REST + CNN | ECM |
| Monzino-AF-T | | | |
| Se_{Dur} | 99.89 | 97.21 | 95.82 |
| PPV_{Dur} | 94.99 | 95.20 | 93.38 |
| Se_{Epi} | 85.71 | 85.71 | 78.57 |
| PPV_{Epi} | 13.19 | 43.08 | 63.38 |
| GISSI 1-T | | | |
| FD/hr | 4.55 | 0.64 | 16.28 |

Table 7 provides duration- and episode-performance on the Monzino-AF-T dataset. Results for the VERITAS-REST and the VERITAS-REST+CNN correspond to performance achieved before and after identification of false AF segments with CNN, respectively.

A sample-by-sample labeling was generated based on the VERITAS-REST+CNN, and its performance on the Monzino-AF-T was evaluated following the EC57 standard and reported in Table 7. In this experimental example, the VERITAS-REST+CNN only missed 2 BAF episodes of 4.5 and 7 seconds each, which agrees with FIG. 14. Additionally, the FD/hr ratio on the GISSI 1-T was 4.55 and 0.64 for the VERITAS-REST and VERITAS-REST+CNN, respectively.

The ECM-image methodology was also implemented, and applied it to the GISSI 1-T and the Monzino-AF-T using lead II, as it was the best performing lead reported by the authors. In this experimental example, the ECM-image method resulted in FD/hr ratio of 16.28 on the GISSI 1-T, and for the Monzino-AF-T the EC57 metrics resulted in Se_{Dur}=95.82, PPV_{Dur}=93.38, Se_{Epi}=78.57 and PPV_{Epi}=63.38. The ECM-image method only left undetected one of the 8 AF episodes shorter than 30 seconds in the Monzino-AF-T database. However it produced a much higher FD/hr ratio than the VERITAS-REST+CNN in this example.

### Discussion

The experimental example presents a new approach for detecting BAF episodes on continuous ECG recordings. The detection is made based on 10-second moving windows with 5-second overlapping over the full continuous recording. The 10-second segments are then analyzed with a commercial 12-lead resting ECG algorithm (VERITAS-REST) which provides rhythm statements. Finally, all rhythm statements were merged to generate a continuous labeling for the whole ECG recording. This method was first tested on continuous ECG recordings for which the duration of the BAF was artificially controlled. Results from this analysis demonstrate that the VERITAS-REST can indeed be used for the detection of BAF episodes embedded in continuous ECG signals, see FIG. 14. This figure indicates that VERITAS-REST detected BAF episodes longer than 5-second with sensitivity higher than 80% and detected all episodes longer than 10 seconds. The method was also tested on continuous ECG recordings from two databases. For the Monzino-AF-T database, that is rich in AF episodes, results from the experimental example showed good Se_{Epi}=85.71% but a low PPV_{Epi}=13.19% mainly due to a high number of false detections when signals were corrupted with motion artifact, see Table 7. The high number of false AF detections was confirmed in the GISSI 1-T database, which contains no AF, where the VERITAS-REST resulted in 4.55 FD/hr in the experimental example. For this metric, the false AF detections per hour of data produced by the clear commercial algorithm VERITAS Arrhythmia/ST AnalysisTM was used as a comparison benchmark, which produced 0.19 FD/hr. To reduce the number of false detections produced by the VERITAS-REST, a CNN was then trained to identify segments that were incorrectly classified as AF by VERITAS-REST based on spectrogram images. To avoid overfitting, the CNN was validated and tested with mutually exclusive datasets randomly selected for which performance on the testing dataset is above 88% for all metrics, indicating that the CNN was capable of identifying 10-second ECG segments that are likely to be misclassified as AF. Later the output of the VERITAS-REST+CNN were mapped to a sample-by-sample labeling and evaluated following the EC57 standard. For the VERITAS-REST+CNN, episode positive predictive value improved from 13.19% to 43.08% on the Monzino-AF-T database and the number of false detections went down from 4.55 to 0.64 per hour on the GISSI 1-T database while episode sensitivity remained on the Monzino-AF-T database in the experimental example, indicating that detection of BAF episodes is not affected by the CNN.

Four research groups have previously investigated the impact of noise identification to improve AF detection based on rhythm irregularity. Oster and Clifford (2015) analyzed how signal-to-noise ratio affects beat identification and consequently deteriorates AF detection. The study was done by simulating and adding motion artifact with different levels of signal-to-noise ratio to recordings in the Long-Term Atrial Fibrillation database (Goldberger et al. (2000), Circulation 101, e215-e220). A few years later, Taji et al., IEEE Transactions on Instrumentation and Measurement 67, 1124-31 implemented a deep belief network to identify ECG segments with unreliable QRS detections. For this purpose, the authors added motion artifact from the MIT-BIH noise stress database (Goldberger et al. (2000), Circulation 101, e215-e220) to recordings in the AFDB and showed that AF detection improves after excluding unreliable QRS detections.

In another research group, Bashar et al., IEEE Access 7, 88357-88368, focused on discriminating between ECG and non-ECG waveforms in the Medical Information Mart for Intensive Care database (Johnson, A. E., et al. (2016), Scientific Data 3, 160035) using both time and spectral-domain properties. The authors showed AF detection is better when non-ECG waveforms are excluded from analysis. Finally, Halvaei et al., Frontiers in Physiology 12, 1-10 used annotated recordings from the StrokeStop I database (Svennberg et al. (2015), Circulation 131, 2176-2184) for training a CNN to identify QRS misdetections trigger by transient noise, achieving a reduction in false AF detections without any loss in sensitivity. Differently than the current experimental example, previous studies may have not investigated detection of BAF episodes since rhythm-based AF detectors usually requires long-detection windows to achieve optimal performance. Nevertheless, the present disclosure also indicates that signal-quality evaluation is highly recommended to improve AF detection.

| | | Detection | | | |
|---|---|---|---|---|---|
| Author | Methodology | length | | Se_{Dur} | AFDB usage |
| Petrenas et al. (2015), | Rhythm irregularity | 15 | seconds | 96.6 | All recordings used for |
| Computers in biology and | | | | | validation |
| medicine 65, 184-191 | | | | | |
| Ladavich and Ghoraani | P-wave absence | 7 | beats | 98.9 | 10 min from each recording |
| (2015), Biomedical Signal | | | | | used for training, |
| Processing and Control 18, | | | | | remaining data for |
| 274-281 | | | | | validation |
| Rodenas et al. (2015), | Wavelet entropy | 10 | beats | 96.5 | Subsegments of AFDB |
| Entropy 17, 6179-6199 | on single | | | | were used for training and |
| | beat for P-wave | | | | validation |
| | detection | | | | |
| Mousavi et al. (2020), | BRNN analyzing | 10 | beats | 90.5 | 10-fold cross validation |
| HAN-ECG: An | sequences of ECG | | | | with mutually exclusive |
| interpretable atrial | heartbeats | | | | data in training and |
| fibrillation | | | | | validation sets |
| detection model using | | | | | |
| hierarchical attention | | | | | |
| networks. arXiv preprint | | | | | |
| arXiv: 2002.05262 | | | | | |
| Asgari et al. (2015), | Stationary wavelet | 10 | seconds | 96.6 | 2-fold cross validation with |
| Computers in Biology and | transform on raw | | | | training and validation sets |
| Medicine 60, 132-142 | ECG segments | | | | from same patients |
| Xia et al. (2018), | CNN analyzing | 5 | seconds | 98.3 | 10-fold cross validation |
| Computers in biology and | time-frequency | | | | with training and |
| medicine 93, 84-92 | images from raw | | | | validation sets from same |
| | ECG segments | | | | patients |
| He et al. (2018), | CNN analyzing | 5 | beats | 99.4 | Data from one fourth of |
| Frontiers | time-frequency | | | | patients used for |
| in physiology | images from raw | | | | validation and |
| 9, 1206 | ECG segments | | | | remaining |
| | | | | | for training |
| Jin et al. (2020), | TACLSTMNN | 5 | beats | 97.5 | Data from four patients |
| Knowledge-Based | analyzing | | | | used for validation and |
| Systems | scalogram images | | | | remaining for training |
| 193, 105460 | from raw ECG | | | | |
| | segments | | | | |

Table 8 shows brief atrial fibrillation detectors validated on the MIT-BIH Atrial Fibrillation database (AFDB). “BRNN” refers to bidirectional recurrent neural network. “TACLSTMNN” refers to twin attentional convolutional long short-term memory neural networks.

Finally, the AF detection algorithm based on ECM methodology described in Salinas-Martinez et al. (2021) was implemented and applied to the same databases as a comparison. In the experimental example, the ECM method worked 98.65%, but produced many false detections on the GISSI 1-T database (16.28 FD/hr). Comparison with other methodologies for BAF detection is not straight forward as most of the techniques were validated with the AFDB. Table 8 summarizes performance of state-of-the-art methodologies for BAF detection validated using the AFDB. It is worth mentioning that only Petrenas et al. (2015) used all recordings in the AFDB for validations, the rest of the methodologies performed validation using subsets of the AFDB. Furthermore, only Petrenas et al. (2015), Mousavi et al. (2020), He et al. (2018), and Jin et al. (2020) used mutually exclusive data for training and validation to avoid biasing performance. Compared to detectors in Table 8, the VERITAS-REST is the best performing for Se_{Dur}, and VERITAS-REST+CNN the third best performing. However, the performance of the detectors were evaluated on different datasets, because the AFDB only contains 2 leads and the method described in this Experimental Example utilizes 8 independent leads (standard leads I, II, V1-V6). Further, it is not clear from the references in Table 8 how these detectors performed on the 58 AF episodes shorter than 30-second Salinas-Martinez et al. (2021) present in the AFDB, recalling that high Se_{Dur} does not guarantee detection of BAF episodes shorter than 30-second. Unfortunately, the AFDB cannot be used to test the proposed methodology as it only contains 2 leads.

It is worth recalling that high Se_{Dur} does not guarantee detection of AF episodes shorter than 30 seconds. However, the AF detection algorithm was implemented based on ECM methodology described in Salinas-Martinez et al. (2021) and applied it to the Monzino-AF-T and the GISSI 1-T databases. The ECM method achieved episode sensitivity of 78.57% and positive predictive value of 63.38% on the Monzino-AF-T 334 database and produced 16.28 FD/hr on the GISSI 1-T database.

### EXAMPLE CLAUSES

1. A method to detect episodes of atrial fibrillation (AF), the method including: identifying ECG data indicative of an electrocardiogram (ECG) of a subject, the ECG being detected by a wearable device worn by the subject; dividing the ECG data into multiple segments including a first segment and a second segment; determining, using a classifier, a first label indicating that the first segment is indicative of AF; generating a first image that is indicative of the first segment; determining, by inputting the first image into a trained convolutional neural network (CNN), that the first label is a false positive; determining, using the classifier, a second label indicating that the second segment is indicative of AF; generating a second image that is indicative of the second segment; determining, by inputting the second image into the trained CNN, that the second label is a true positive; and outputting the second segment without outputting the first segment.

2. The method of clause 1, wherein identifying the ECG includes detecting the ECG by a Holter monitor worn by the subject.

3. The method of clause 1 or 2, wherein the ECG includes twelve leads.

4. The method of any one of clauses 1 to 3, wherein the ECG includes less than twelve leads.

5. The method of any one of clauses 1 to 4, wherein the first segment and the second segment have a length that is greater than or equal to about 5 seconds and less than or equal to about 30 seconds.

6. The method of any one of clauses 1 to 5, wherein determining, using the classifier, the first label indicating that the first segment is indicative of AF includes: detecting first beats in the first segment; identifying first beat-to-beat intervals defined between the first beats; determining whether a first p wave is present in the first segment; and determining that the first segment is indicative of AF based on the first beat-to-beat intervals and whether the first p wave is present in the first segment, and wherein determining, using the classifier, the second label indicating that the second segment is indicative of AF includes: detecting second beats in the second segment; identifying second beat-to-beat intervals defined between the second beats; determining whether a second p wave is present in the second segment; and determining that the second segment is indicative of AF based on the second beat-to-beat intervals and determining whether the second p wave is present in the second segment.

7. The method of any one of clauses 1 to 6, wherein generating the first image that is indicative of the first segment includes generating a first spectrogram of the first segment, and wherein generating the second image that is indicative of the second segment includes generating a second spectrogram of the second segment.

8. The method of any one of clauses 1 to 7, wherein the trained CNN includes: a first convolutional block including a first convolutional layer, a first batch normalization layer, a first rectified linear unit (ReLu) activation layer, and a first max pooling layer; a second convolutional block connected in series to the first convolutional block, the second convolutional block including a second convolutional layer, a second batch normalization layer, a second ReLu activation layer, and a second max pooling layer; a third convolutional block connected in series to the second convolutional block, the third convolutional block including a third convolutional layer, a third batch normalization layer, and a third ReLu activation layer; and a softmax layer connected in series to the third convolutional block.

9. The method of any one of clauses 1 to 8, further including: generating the trained CNN by optimizing parameters of the CNN based on training data, the training data including: training images indicative of other ECGs of other subjects; and ground truth labels indicating whether the other ECGs depict AF.

10. The method of any one of clauses 1 to 9, wherein determining, using the classifier, a second label indicating whether the second segment is indicative of AF includes: detecting a brief episode of AF in the second segment, the brief episode of AF occurring for greater than or equal to 5 seconds and less than or equal to 30 seconds.

11. The method of any one of clauses 1 to 10, further including: determining an AF burden based on the second label; and outputting an indication of the AF burden.

12. A system, including: a processor; and memory storing instructions that, when executed by the processor, cause the system to perform the method of any one of clauses 1 to 11.

13. The system of clause 12, further including: a wearable or hand-held device configured to detect the ECG and to generate the ECG data.

14. The system of clause 12 or 13, further including: a transceiver configured to receive a signal indicative of the ECG data from a wearable device.

15. A method, including: identifying data indicative of measurements of a physiological parameter of a subject over a time period; dividing the data into multiple segments including a first segment and a second segment; determining, by detecting at least one first characteristic of the first segment, a first label indicating whether the first segment is indicative of a condition; generating a first image that is indicative of the first segment; determining, by inputting the first image into a trained machine learning (ML) model, that the first label is inaccurate; determining, by detecting at least one second characteristic of the second segment, a second label indicating whether the second segment is indicative of the condition; generating a second image that is indicative of the second segment; determining, by inputting the second image into the trained ML model, that the second label is accurate; and outputting an indication of whether the subject has the condition based on the second label.

16. The method of clause 15, wherein the physiological parameter includes an electrocardiogram (ECG), a plethysmograph, a capnograph, an electroencephalogram (EEG), or an electromyograph (EMG).

17. The method of clause 15 or 16, wherein the condition includes an arrhythmia or a seizure.

18. The method of any one of clauses 15 to 17, wherein the condition includes atrial fibrillation (AF), atrial flutter, ventricular tachycardia, supraventricular tachycardia, or an atrioventricular block.

19. The method of any one of clauses 15 to 18, wherein the measurements of the physiological parameter of the subject are generated by a wearable device worn by the subject.

20. The method of any one of clauses 15 to 19, wherein the first segment includes at least one artifact associated with motion of the subject when the measurements of the physiological parameter were obtained.

21. The method of any one of clauses 15 to 20, wherein generating the first image that is indicative of the first segment includes generating a first spectrogram of the first segment, and wherein generating the second image that is indicative of the second segment includes generating a second spectrogram of the second segment.

22. The method of any one of clauses 15 to 21, wherein the trained ML model includes a CNN.

23. The method of clause 22, wherein the CNN includes: a first block including a first convolutional layer, a first batch normalization layer, a first rectified linear unit (ReLu) activation layer, and a first max pooling layer; a second block connected in series to the first block, the second block including a second convolutional layer, a second batch normalization layer, a second ReLu activation layer, and a second max pooling layer; a third block connected in series to the second block, the third block including a third convolutional layer, a third batch normalization layer, and a third ReLu activation layer; and a softmax layer connected in series to the third block.

24. The method of clause 22 or 23, further including: generating the trained ML model by optimizing parameters of an untrained ML model based on training data, the training data including: training images indicative of other measurements of the physiological parameter of other subjects; and ground truth labels indicating whether the other measurements indicate the condition.

25. The method of any one of clauses 15 to 24, wherein identifying the data includes receiving a signal indicating the data from an external device.

26. The method of any one of clauses 15 to 25, wherein identifying the data includes generating the data by detecting the physiological parameter.

27. A system, including: a processor; and memory storing instructions that, when executed by the processor, cause the system to perform the method of any one of clauses 15 to 26.

28. A monitoring system, including: a wearable device configured to generate physiological parameter data by detecting a physiological parameter of a subject wearing the wearable device at a sampling rate over a time period; and a computing system including at least one processor configured to: generate segments by dividing the physiological parameter data; generate classifications of the segments using a classifier, the classifications indicating whether the segments are indicative of a condition; generate images by generating spectrograms of the segments; determine, by inputting the images into a trained machine learning (ML) model, that the segments include one or more misclassified segments and one or more correctly classified segments; and output one or more of the classifications corresponding to the one or more correctly classified segments.

29. The monitoring system of clause 28, wherein the time period is greater than one minute and an example segment among the segments corresponds to a time interval of less than 30 seconds.

30. The monitoring system of clause 28 or 29, wherein the physiological parameter includes an electrocardiogram (ECG), a plethysmograph, a capnograph, an electroencephalogram (EEG), or an electromyograph (EMG).

31. The monitoring system of any one of clauses 28 to 30, wherein the trained ML model includes a convolutional neural network (CNN).

32. The monitoring system of any one of clauses 28 to 31, wherein the at least one processor is further configured to output the one or more correctly classified segments.

### CONCLUSION(S)

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

The present document includes references to various other patent documents, articles, and other published documents, each of which is incorporated by reference herein in its entirety.

Although the subject matter has been described in language specific to structural features and/or methodological acts, it is to be understood that the subject matter defined in the appended claims is not necessarily limited to the specific features or acts described.

What is claimed is:

1. A method to detect episodes of atrial fibrillation (AF), the method comprising:
identifying electrocardiogram (ECG) data indicative of an ECG of a subject, the ECG being detected by a wearable device worn by the subject or a hand-held device;
dividing the ECG data into multiple segments comprising a first segment and a second segment;
determining, using a classifier, a first label indicating that the first segment is indicative of AF;
generating a first image that is indicative of the first segment;
determining, by inputting the first image into a trained convolutional neural network (CNN), that the first label is a false positive;
determining, using the classifier, a second label indicating that the second segment is indicative of AF;
generating a second image that is indicative of the second segment;
determining, by inputting the second image into the trained CNN, that the second label is a true positive; and
outputting the second segment without outputting the first segment.

2. The method of claim 1, wherein identifying the ECG comprises detecting the ECG by a Holter monitor worn by the subject.

3. The method of claim 1, wherein the ECG comprises twelve leads or fewer than twelve leads.

4. The method of claim 1, wherein the first segment and the second segment each have a length that is greater than or equal to about 5 seconds and less than or equal to about 30 seconds.

5. The method of claim 1, wherein determining, using the classifier, the first label indicating that the first segment is indicative of AF comprises:
detecting first beats in the first segment;
identifying first beat-to-beat intervals defined between the first beats;
determining whether a first p wave is present in the first segment; and
determining that the first segment is indicative of AF based on the first beat-to-beat intervals and whether the first p wave is present in the first segment, and wherein determining, using the classifier, the second label indicating that the second segment is indicative of AF comprises:
detecting second beats in the second segment;
identifying second beat-to-beat intervals defined between the second beats;
determining whether a second p wave is present in the second segment; and
determining that the second segment is indicative of AF based on the second beat-to-beat intervals and determining whether the second p wave is present in the second segment.

6. The method of claim 1, wherein generating the first image that is indicative of the first segment comprises generating a first spectrogram of the first segment, and wherein generating the second image that is indicative of the second segment comprises generating a second spectrogram of the second segment.

7. The method of claim 1, wherein the trained CNN comprises:
a first convolutional block comprising a first convolutional layer, a first batch normalization layer, a first rectified linear unit (ReLu) activation layer, and a first max pooling layer;
a second convolutional block connected in series to the first convolutional block, the second convolutional block comprising a second convolutional layer, a second batch normalization layer, a second ReLu activation layer, and a second max pooling layer;
a third convolutional block connected in series to the second convolutional block, the third convolutional block comprising a third convolutional layer, a third batch normalization layer, and a third ReLu activation layer; and
a softmax layer connected in series to the third convolutional block.

8. The method of claim 1, further comprising:
generating the trained CNN by optimizing parameters of the CNN based on training data, the training data comprising:
training images indicative of other ECGs of other subjects; and
ground truth labels indicating whether the other ECGs depict AF.

9. The method of claim 1, wherein determining, using the classifier, a second label indicating whether the second segment is indicative of AF comprises:
detecting a brief episode of AF in the second segment, the brief episode of AF occurring for greater than or equal to 5 seconds and less than or equal to 30 seconds.

10. The method of claim 1, further comprising:
determining an AF burden based on the second label; and
outputting an indication of the AF burden.

11. A method, comprising:
identifying data indicative of measurements of a physiological parameter of a subject over a time period;
dividing the data into multiple segments comprising a first segment and a second segment;
determining, by detecting at least one first characteristic of the first segment, a first label indicating whether the first segment is indicative of a condition;
generating a first image that is indicative of the first segment;
determining, by inputting the first image into a trained machine learning (ML) model, that the first label is inaccurate;
determining, by detecting at least one second characteristic of the second segment, a second label indicating whether the second segment is indicative of the condition;
generating a second image that is indicative of the second segment;
determining, by inputting the second image into the trained ML model, that the second label is accurate; and
outputting an indication of whether the subject has the condition based on the second label.

12. The method of claim 11, wherein the physiological parameter comprises an electrocardiogram (ECG), a plethysmograph, a capnograph, an electroencephalogram (EEG), or an electromyograph (EMG), or
wherein the condition comprises an arrhythmia a seizure, atrial fibrillation (AF), atrial flutter, ventricular tachycardia, supraventricular tachycardia, or an atrioventricular block.

13. The method of claim 11, wherein the measurements of the physiological parameter of the subject are generated by a wearable device worn by the subject or a hand-held device.

14. The method of claim 11, wherein the first segment comprises at least one artifact associated with motion of the subject when the measurements of the physiological parameter were obtained.

15. The method of claim 11, wherein generating the first image that is indicative of the first segment comprises generating a first spectrogram of the first segment, and wherein generating the second image that is indicative of the second segment comprises generating a second spectrogram of the second segment.

16. The method of claim 11, wherein the trained ML model comprises a convolutional neural network (CNN) that comprises:
a first block comprising a first convolutional layer, a first batch normalization layer, a first rectified linear unit (ReLu) activation layer, and a first max pooling layer;
a second block connected in series to the first block, the second block comprising a second convolutional layer, a second batch normalization layer, a second ReLu activation layer, and a second max pooling layer;
a third block connected in series to the second block, the third block comprising a third convolutional layer, a third batch normalization layer, and a third ReLu activation layer; and
a softmax layer connected in series to the third block.

17. The method of claim 11, further comprising:
generating the trained ML model by optimizing parameters of an untrained ML model based on training data, the training data comprising:
training images indicative of other measurements of the physiological parameter of other subjects; and
ground truth labels indicating whether the other measurements indicate the condition.

18. The method of claim 11, wherein identifying the data comprises:
receiving a signal indicating the data from an external device; or
generating the data by detecting the physiological parameter.

19. A monitoring system, comprising:
a wearable device configured to generate physiological parameter data by detecting a physiological parameter of a subject wearing the wearable device at a sampling rate over a time period; and
a computing system comprising at least one processor configured to:
generate segments by dividing the physiological parameter data;
generate classifications of the segments using a classifier, the classifications indicating whether the segments are indicative of a condition;
generate images by generating spectrograms of the segments;
determine, by inputting the images into a trained machine learning (ML) model, that the segments comprise one or more misclassified segments and one or more correctly classified segments; and
output one or more of the classifications corresponding to the one or more correctly classified segments.

20. The monitoring system of claim 19, wherein the time period is greater than one minute and an example segment among the segments corresponds to a time interval of less than 30 seconds;
wherein the physiological parameter comprises an electrocardiogram (ECG), a plethysmograph, a capnograph, an electroencephalogram (EEG), or an electromyograph (EMG);
wherein the trained ML model comprises a convolutional neural network (CNN); or
wherein the at least one processor is further configured to output the one or more correctly classified segments.
